(19) United States
(12) Patent Application Publication
(10) Pub. No.: US 2025/0375459 A1
(43) Pub. Date: Dec. 11, 2025

(54) METHODS FOR AMELIORATING COGNITIVE IMPAIRMENT USING BILE ACID DERIVATIVES

(71) Applicant: INTERCEPT PHARMACEUTICALS, INC., Morristown, NJ (US)

(72) Inventors: David E. JONES, Newcastle upon Tyne (GB); Fiona OAKLEY, Newcastle upon Tyne (GB)

(21) Appl. No.: 18/878,533

(22) PCT Filed: Jun. 30, 2023
(86) PCT No.: PCT/US2023/026759

§ 371 Date: Dec. 23, 2024

Related U.S. Application Data
(60) Provisional application No. 63/358,088, filed on Jul. 1, 2022.

(51) Int. Cl. A61K 31/575; A61P 25/28

(52) U.S. Cl. CPC A61K 31/575; A61P 25/28

(57) ABSTRACT

The disclosure relates to methods of using bile acid derivatives for the treatment and/or prevention of cognitive disorders, diseases, or conditions.

### RELATED APPLICATION

This application claims priority to, and the benefit of, U.S. Application No. 63/358,088, filed Jul. 1, 2022, the entire contents of which are incorporated herein by reference.

### BACKGROUND

Mild cognitive impairment is characterized by deficits in memory, language, and/or other essential cognitive functions. The condition can evolve towards dementia, which is characterized by a global deterioration of cognitive abilities to an extent that interferes with daily life. Cognitive impairment is one of the most important non-motor symptoms in Parkinson's disease (PD) and has a considerable impact on both patients and their caregivers. The development of PD dementia (PDD) is a frequent and distressing complication of the disease, with a cumulative incidence approaching 80% in community-based studies (Aarsland, D., et al. Prevalence and characteristics of dementia in Parkinson disease: an 8-year prospective study. Arch Neurol. 2003 March; 60(3):387-92; Buter, T. C., et al. Dementia and survival in Parkinson disease: a 12-year population study. Neurology. 2008 Mar. 25; 70(13):1017-22; Helv, M. A., et al. The Sydney multicenter study of Parkinson's disease: the inevitability of dementia at 20 years. Mov Disord. 2008 Apr. 30; 23(6):837-44). Importantly, mild cognitive impairment (MCI) is a risk factor for the development of PDD and is present in over 40% of patients with early disease (Yarnall, A. J., et al. Characterizing mild cognitive impairment in incident Parkinson disease: the ICICLE-PD study. Neurology 2014 Jan. 28; 82(4):308-16). The implication of this observation is that PD-MCI represents an important early stage in the PDD pathways giving rise to the hypothesis that effective treatment of patients with PD-MCI may significantly reduce the risk of progression to PDD. Where PDD does develop, the impact goes beyond significant cognitive dysfunction. PDD is associated with a twofold increase in mortality (Levy, G., et al. The association of incident dementia with mortality in PD. Neurology. 2002 Dec. 10; 59(11):1708-13), a loss of independence (Aarsland, D., et al. Predictors of nursing home placement in Parkinson's disease: a population-based, prospective study. J Am Geriatr Soc. 2000 August; 48(8):938-42), increased caregiver strain (Aarsland, D., et al. Neuropsychiatric symptoms in patients with Parkinson's disease and dementia: frequency, profile and associated care giver stress. J Neurol Neurosurg Psychiatry. 2007 January; 78(1):36-42.), and increased healthcare costs (Vossius, C., et al. The economic impact of cognitive impairment in Parkinson's disease. Mov Disord. 2011 July; 26(8):1541-4), the latter largely due to the cost of institutionalization.

Obeticholic acid (OCA) is a modified bile acid and famesoid X receptor (FXR) agonist. Based on preclinical data, OCA can reverse cognitive dysfunction in cholestasis (e.g., PBC) when given early in the disease course (Gee et al., Journal of Hepatology (2019) 70, e75-e76).

There is a need for developing products which show improved activity in the treatment and/or prevention of neurological disorders accompanied by cognitive deficit or impairment, or neurodegenerative diseases. There are currently no cognitive treatments for PD, and the only approved agent for PDD is rivastigmine (sold under the trade name EXELON among others). Therefore, there is a major market gap for an effective alternative cognitive enhancer in these patient groups and there is a substantial unmet clinical need for new methods of treating and improving the quality of life in patients having cognitive impairment and slowing the progression of dementia.

### SUMMARY

In one aspect, the present application provides a method of ameliorating, mitigating, treating, or preventing cognitive impairment in a subject, comprising administering to the subject at least one therapeutically effective amount of a compound of formula (I):

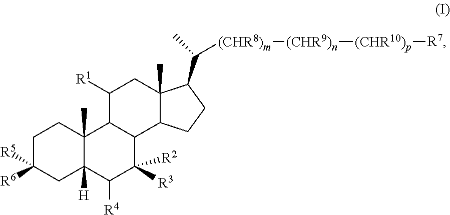

or a pharmaceutically acceptable salt, ester, solvate, or amino acid conjugate thereof, wherein:
R¹ is H, OH, alkoxy, or oxo;
R² is H, OH, halogen, or alkyl optionally substituted with one or more halogen or OH, or R² and R³ taken together with the carbon atom to which they are attached form a carbonyl;
R³ is H, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, or halogen, or R⁵ and R⁶ taken together with the carbon atom to which they are attached form a carbonyl;
R⁷ is OH, OSO₃H, SO₃H, 2NH₂, SO₂NH₂, OPO₃H₂, PO₃H₂, CO₂H, C(O)NHOH, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, or 2,4-difluoro-3-hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
m is 0, 1, or 2;
n is 0 or 1; and
p is 0 or 1.

In some embodiments, the compound of formula (I) is a compound of formula (A):

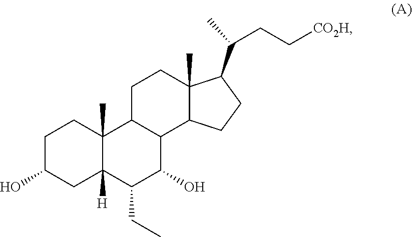 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or pharmaceutically acceptable salt, ester, solvate, or amino acid conjugate thereof.

In some embodiments, the compound of formula (I) is a compound of formula (A).

In some embodiments, the compound of formula (I) is a compound of formula (B):

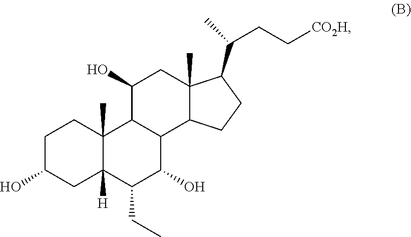 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

or pharmaceutically acceptable salt, ester, solvate, or amino acid conjugate thereof.

In some embodiments, the compound of formula (I) is a compound of formula (B).

In some embodiments, the compound of formula (I) is a compound of formula (C):

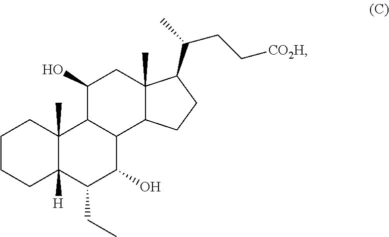 [SMILES: CC[C@H]1[C@@H](O)C2C3CC[C@H]([C@H](C)CCC(=O)O)[C@@]3(C)C[C@H](O)C2[C@@]2(C)CCCC[C@@H]12]

or pharmaceutically acceptable salt, ester, solvate, or amino acid conjugate thereof.

In some embodiments, the compound of formula (I) is a compound of formula (C).

In some embodiments, the compound of formula (I) is a compound of formula (D):

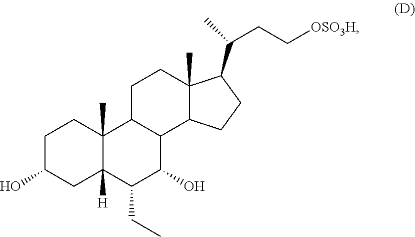

or pharmaceutically acceptable salt, ester, solvate, or amino acid conjugate thereof.

In some embodiments, the compound of formula (I) is a compound of formula (D).

In some embodiments, the cognitive impairment is associated with a neurological disease.

In some embodiments, the neurological disease is Parkinson's disease.

In some embodiments, the neurological disease is Parkinson's disease dementia.

In some embodiments, wherein the neurological disease is mild cognitive impairment in Parkinson's disease PD dementia (PD-MCI).

In some embodiments, the effective amount of the compound of formula (I) is between about 1 mg to and about 50 mg.

In some embodiments, the effective amount of the compound of formula (I) is between about 1 mg to and about 25 mg.

In some embodiments, the effective amount of the compound of formula (I) is about 25 mg.

In some embodiments, the effective amount of the compound of formula (I) is administered about every day, about every two days, about every three days, or about every week.

In some embodiments, the effective amount of the compound of formula (I) is administered daily.

In another aspect, the present application provides a method of reversing cellular senescence associated with Parkinson's Disease in the neurons of a subject, comprising administering to the subject at least one therapeutically effective amount of a compound of formula (I) or a pharmaceutically acceptable salt, solvate, or amino acid conjugate thereof.

In another aspect, the present application provides a method of ameliorating, mitigating, treating, or preventing telomere dysfunction associated with Parkinson's Disease in the neurons of a subject, comprising administering to the subject at least one therapeutically effective amount of a compound of formula (I) or a pharmaceutically acceptable salt, solvate, or amino acid conjugate thereof.

In another aspect, the present application provides a use of a compound of formula (I) or a pharmaceutically acceptable salt, solvate, or amino acid conjugate thereof, for ameliorating, mitigating, treating, or preventing cognitive impairment in a subject.

In another aspect, the present application provides a use of a compound of formula (I) or a pharmaceutically acceptable salt, solvate, or amino acid conjugate thereof, for the manufacture of a medicament for ameliorating, mitigating, treating, or preventing cognitive impairment in a subject.

In another aspect, the present application provides a compound of formula (I) or a pharmaceutically acceptable salt, ester, solvate, or amino acid conjugate thereof, for use in ameliorating, mitigating, treating, or preventing cognitive impairment in a subject.

In another aspect, the present application provides a kit for treating, ameliorating, or mitigating cognitive impairment in a subject, comprising at least one therapeutically effective amount of a compound of formula (I) or a pharmaceutically acceptable salt, ester, solvate, or amino acid conjugate thereof, and instructions for use.

In another aspect, the present application provides a pharmaceutical composition for treating, ameliorating, or mitigating cognitive impairment in a subject comprising at least one therapeutically effective amount of a compound of formula (I) or a pharmaceutically acceptable salt, ester, solvate, or amino acid conjugate thereof, and at least one pharmaceutically acceptable excipient.

Unless otherwise defined, all technical and scientific terms used herein have the same meaning as commonly understood by one of ordinary skill in the art to which this application belongs. In the case of conflict, the present specification, including definitions, will control. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art to the present application. In addition, the materials, methods, and examples are illustrative only and are not intended to be limiting. Other features and advantages of the present application will be apparent from the following detailed description and claims.

### BRIEF DESCRIPTION OF THE DRAWINGS

FIG. 1 is an illustration of an aged mice cohort study plan.

FIG. 2 is a bar graph showing the results of Y-maze testing on aged mice treated with ursodeoxycholic acid (URSO), obeticholic acid (OCA), or control at 11 and 14 months, according to the cohort study plan in FIG. 1.

FIG. 3 is a bar graph showing immunofluorescence analysis of aged mice treated with obeticholic acid (OCA), ursodeoxycholic acid (URSO), or no supplement (wild type or WT) ad libitum each at 15 months (15 mnth) vs. 9 months (9 mnth), according to the cohort study plan in FIG. 1.

### DETAILED DESCRIPTION

The present application relates to the methods of treatment and/or prevention of neurological disorders accompanied by cognitive deficit or impairment, or neurodegenerative diseases using bile acid derivatives, or their compositions as described herein.

The present application is directed to methods of ameliorating, mitigating, treating, or preventing cognitive diseases, disorders or conditions, using a compound of formula (I):

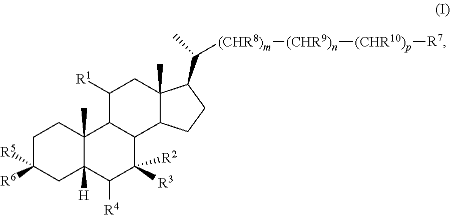

or a pharmaceutically acceptable salt, ester, solvate, or amino acid conjugate (such as, e.g., glycine, taurine or sarcosine conjugate) thereof, wherein:
R¹ is H, OH, alkoxy, or oxo;
R² is H, OH, halogen, or alkyl optionally substituted with one or more halogen or OH, or R² and R³ taken together with the carbon atom to which they are attached form a carbonyl;
R³ is H, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, or halogen, or R⁵ and R⁶ taken together with the carbon atom to which they are attached form a carbonyl;
R⁷ is OH, OSO₃H, SO₃H, OSO₂NH₂, SO₂NH₂, OPO₃H₂, PO₃H₂, CO₂H, C(O)NHOH, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, or 2,4-difluoro-3-hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
m is 0, 1, or 2;
n is 0 or 1; and
p is 0 or 1.

The present disclosure relates to the methods of amelioration, mitigation, treatment and/or prevention of FXR-mediated cognitive diseases, disorders or conditions, using a compound of formula (I) or a pharmaceutically acceptable salt, ester, solvate, or amino acid conjugate thereof.

In some embodiments, the present application is directed to the use of a compound of formula (I), or a pharmaceutically acceptable salt, ester, solvate, or amino acid conjugate (such as, e.g., glycine, taurine or sarcosine conjugate) thereof, for ameliorating, mitigating, treating, or preventing cognitive diseases, disorders, or conditions.

In some embodiments, the present application is directed to the use of a compound of formula (I), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing cognitive diseases, disorders, or conditions.

In some embodiments, the present application is directed to a compound of formula (I), or a pharmaceutically acceptable salt, ester, solvate, or amino acid conjugate (such as, e.g., glycine, taurine or sarcosine conjugate) thereof, for use in ameliorating, mitigating, treating, or preventing cognitive diseases, disorders, or conditions.

In some embodiments, the present application is directed to a kit for ameliorating, mitigating, treating, or preventing cognitive diseases, disorders, or conditions, comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing cognitive diseases, disorders, or conditions, comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

The compounds of formula (I) disclosed herein can be prepared by conventional methods (e.g., as described in U.S. Pat. Nos. 7,932,244; 7,994,352; 9,611,289; 9,777,038; 10,202,414; 10,155,787; 10,174,073; 10,815,267; 11,034,717; and 11,066,437).

In some embodiments, the present application is directed to methods of ameliorating, mitigating, treating, or preventing cognitive diseases, disorders or conditions, using a compound of formula (I) or a pharmaceutically acceptable salt, ester, solvate, or amino acid conjugate thereof, wherein the compound of formula (I) is a compound of formula (A) (obeticholic acid, also known as OCA, INT-747, 6-ethylchenodeoxycholic acid, or 6-ECDCA):

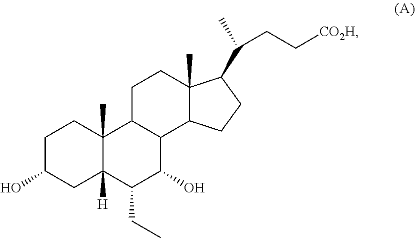 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or a pharmaceutically acceptable salt, ester, solvate, or amino acid conjugate (such as, e.g., glycine, taurine or sarcosine conjugate) thereof.

In some embodiments, the present application is directed to methods of ameliorating, mitigating, treating, or preventing cognitive diseases, disorders or conditions using a compound of formula (I) or a pharmaceutically acceptable salt, ester, solvate, or amino acid conjugate thereof, wherein the compound of formula (I) is a compound of formula (B):

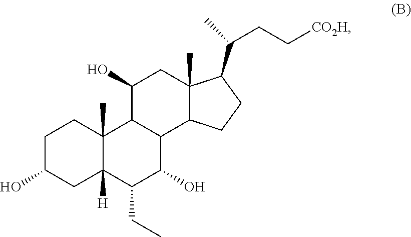 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

or a pharmaceutically acceptable salt, ester, solvate, or amino acid conjugate (such as, e.g., glycine, taurine or sarcosine conjugate) thereof.

In some embodiments, the present application is directed to methods of ameliorating, mitigating, treating, or preventing cognitive diseases, disorders, or conditions using a compound of formula (I) or a pharmaceutically acceptable salt, ester, solvate, or amino acid conjugate thereof, wherein the compound of formula (I) is a compound of formula (C):

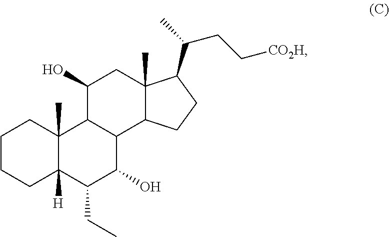 [SMILES: CC[C@H]1[C@@H](O)C2C3CC[C@H]([C@H](C)CCC(=O)O)[C@@]3(C)C[C@H](O)C2[C@@]2(C)CCCC[C@@H]12]

or a pharmaceutically acceptable salt, ester, solvate, or amino acid conjugate (such as, e.g., glycine, taurine or sarcosine conjugate) thereof.

In some embodiments, the present application is directed to methods of ameliorating, mitigating, treating, or preventing cognitive diseases, disorders or conditions using a compound of formula (I) or a pharmaceutically acceptable salt, ester, solvate, or amino acid conjugate thereof, wherein the compound of formula (I) is a compound of formula (D):

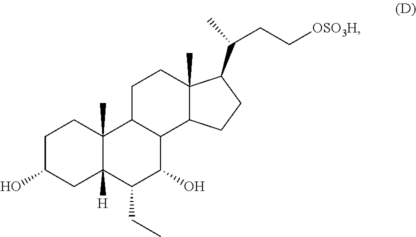

or a pharmaceutically acceptable salt, ester, solvate, or amino acid conjugate (such as, e.g., glycine, taurine or sarcosine conjugate) thereof.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present specification will control. All patents and publications cited in this specification are incorporated herein by reference in their entireties.

All percentages and ratios used herein, unless otherwise indicated, are by weight.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “organ” refers to a differentiated structure (e.g., brain, etc.) consisting of cells and tissues and performing some specific function in an organism. This term also encompasses bodily parts performing a function or cooperating in an activity (e.g., an eye and related structures that make up the visual organs). The term “organ” further encompasses any partial structure of differentiated cells and tissues that is potentially capable of developing into a complete structure (e.g., a lobe).

As used herein, the term “6-ethyl chenodeoxycholic acid”, “6-ECDCA”, “obeticholic acid”, or “OCA” refers to a compound having the chemical structure:

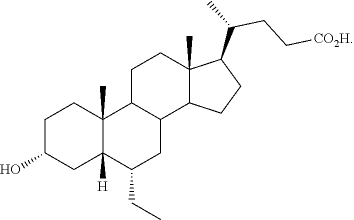 [SMILES: CC[C@H]1CC2C3CC[C@H]([C@H](C)CCC(=O)O)[C@@]3(C)CCC2[C@@]2(C)CC[C@@H](O)C[C@@H]12]

Other chemical names for obeticholic acid include: 3α,7α-dihydroxy-6α-ethyl-5β-cholan-24-oic acid, 6α-ethyl-chenodeoxycholic acid, 6-ethyl-CDCA, 6ECDCA, (3α,5β, 6α,7α)-6-ethyl-3,7-dihydroxycholan-24-oic acid, and INT-747. The CAS registry number for obeticholic acid is 459789-99-2. This term refers to all forms of obeticholic acid, e.g., non-crystalline, crystalline, and substantially pure.

The disclosure also comprehends isotopically labeled obeticholic acid, or pharmaceutically acceptable salts, esters, solvates, or amino acid conjugates thereof, which are identical to those recited in formulae of the disclosure and following, but for the fact that one or more atoms are replaced by an atom having an atomic mass or mass number different from the atomic mass or mass number most commonly found in nature. Examples of isotopes that can be incorporated into obeticholic acid, or pharmaceutically acceptable salts, esters, solvates, or amino acid conjugates thereof include isotopes of hydrogen, carbon, nitrogen, and fluorine, such as ³H, ¹¹C, ¹⁴C, and ¹⁸F.

Obeticholic acid, or pharmaceutically acceptable salts, esters, solvates, or amino acid conjugates thereof that contain the aforementioned isotopes and/or other isotopes of other atoms are within the scope of the present disclosure. Isotopically labeled obeticholic acid, or pharmaceutically acceptable salts, solvates, or amino acid conjugates thereof, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances, isotopically labeled obeticholic acid, or pharmaceutically acceptable salts, solvates, or amino acid conjugates thereof can generally be prepared by carrying out the procedures disclosed in the Schemes and/or in the Examples of the disclosure, by substituting a readily available isotopically labeled reagent for a non-isotopically labeled reagent. In one embodiment, obeticholic acid, or pharmaceutically acceptable salts, solvates, or amino acid conjugates thereof are not isotopically labeled. In one embodiment, deuterated obeticholic acid is useful for bioanalytical assays. In some embodiments, obeticholic acid, or pharmaceutically acceptable salts, solvates, or amino acid conjugates thereof are radiolabeled.

As used herein, the term “pharmaceutically acceptable salt” refers to base addition salts including, but are not limited to, alkali metal salts selected from sodium, lithium, or potassium salt or alkaline earth metal salts selected from calcium or magnesium. Base addition salts further include inorganic and organic amine salts including, but are not limited to, ammonium, methylammonium, ethylammonium, diethylammonium, triethylammonium, lysine, arginine, N-methylglucamine, and choline. Conventional non-toxic salts also include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

“Solvate”, as used herein, refers to a solvent addition form of a compound of formula (A) that contains either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

As used herein, the term “amino acid conjugates” refers to conjugates of a compound of the disclosure with any suitable amino acid. Taurine (—NH(CH₂)₂SO₃H), glycine (—NHCH₂CO₂H), and sarcosine (—N(CH₃)CH₂CO₂H) are examples of amino acid conjugates. Suitable amino acid conjugates of the compounds have the added advantage of enhanced integrity in bile or intestinal fluids. Suitable amino acids include, but are not limited to taurine, glycine, and sarcosine. The amino acid conjugates of the compounds of the disclosure can be prepared according to methods known in the art. For example, a free or protected bile acid or bile acid derivative can be coupled to an amino acid (protected or unprotected), e.g., glycine, sarcosine, or taurine amino acid, using standard peptide coupling conditions (e.g., in the presence of a base (e.g., triethylamine, diisopropyl ethylamine (DIPEA), etc.) and specific coupling reagents, for example, N-Ethoxycarbonyl-2-ethoxy-1,2-dihydroquinoline (EEDQ), 4-(4,6-Dimethoxy-1,3,5-triazin-2-yl)-4-methylmorpholinium chloride (DMTMM), etc.).

In some embodiments described herein, the compositions of the present disclosure (e.g., a composition of a compound of formula (I) (e.g., a compound of formula (A), (B), or (C)) can be metabolized to an amino acid conjugate (e.g., obeticholic acid amino acid conjugate), such as for example, a glycine, taurine, or sarcosine conjugate of the free acid form. Such metabolites can be useful in treating a disease or condition provided herein. In certain instances, production of conjugates can be assessed, monitored, measured, or detected, as described herein during the course of a treatment. In some embodiments, increased levels of the amino acid conjugates (e.g., OCA amino acid conjugates) can result in adjusted dosages of the pharmaceutical compositions described herein (e.g., obeticholic acid composition).

OCALIVA® is an approved drug, which is a tableted form of obeticholic acid. OCALIVA® tablets are supplied in 5 mg and 10 mg strengths for oral administration. OCALIVA® is indicated for the treatment of primary biliary cholangitis (PBC) in combination with ursodeoxycholic acid (UDCA) in adults with an inadequate response to UDCA, or as monotherapy in adults unable to tolerate UDCA.

“Treating” includes any effect, e.g., lessening, reducing, modulating, or eliminating, that results in the improvement of the condition, disease, disorder, etc. “Treating” or “treatment” of a disease state includes arresting the development of the disease state or its clinical symptoms; or relieving the disease state, i.e., causing temporary or permanent regression of the disease state or its clinical symptoms.

“Mitigating” as used herein means to make a symptom or disease less severe or less intense.

“Ameliorating” as used herein means to make a symptom or disease better or more tolerable. Amelioration means an improvement in a patient's condition, or the activity of making an effort to correct, or at least make more acceptable, conditions that are difficult to endure related to patient's conditions.

“Preventing” the disease state includes causing the clinical symptoms of the disease state not to develop in a subject that may be exposed to or predisposed to the disease state but does not yet experience or display symptoms of the disease state.

The term “inhibiting” or “inhibition,” as used herein, refers to any detectable positive effect on the development or progression of a disease or condition. Such a positive effect may include the delay or prevention of the onset of at least one symptom or sign of the disease or condition, alleviation or reversal of the symptom(s) or sign(s) and slowing or prevention of the further worsening of the symptom(s) or sign(s).

“Disease state” means any disease, disorder, condition, symptom, or indication as described herein.

“Cellular Senescence,” as used herein, refers to a condition in which a cell no longer has the ability to proliferate, i.e., has undergone a stable arrest in the cell cycle. In some embodiments, cellular senescence is identified by methods known in the art, e.g., as disclosed in e.g., González-Gualda, E., Baker, A. G., Fruk, L. and Muñoz-Espín, D. (2021), A guide to assessing cellular senescence in vitro and in vivo. FEBS J, 288: 56-80; Hernandez-Segura, Alejandra, et al. “Hallmarks of Cellular Senescence.” Trends in Cell Biology, vol. 28, no. 6, June 2018, pp. 436-453; Bernadotte, Alexandra et al. “Markers of cellular senescence. Telomere shortening as a marker of cellular senescence.” Aging vol. 8, 1 (2016): 3-11. doi: 10.18632/aging.100871; and/or Wang, Audrey S., and Oliver Dreesen. “Biomarkers of Cellular Senescence and Skin Aging.” Frontiers in Genetics, vol. 9, 23 Aug. 2018, the contents of which are incorporated by reference in their entireties for all purposes. In some embodiments, cellular senescence is identified by methods known in the art, e.g. through the analysis of the expression level (e.g. mRNA expression levels and/or protein expression levels) of established markers known to be specifically associated with the senescence state, e.g., Ki67 (downregulated), p16^{INK4a} (upregulated), pRB (upregulated), phospho-pRb (downregulated), p21 (upregulated), p53 (upregulated), phospho-p53 (upregulated), Deleted in Esophageal Cancer 1 (DEC1; BHLHB2; upregulated)), Protein Phosphatase 1 alpha (PPP1A; upregulated), Intracellular Adhesion Molecule 1 (ICAM-1; upregulated), DEP1 (upregulated), Beta 2 Microglobulin (B2MG; upregulated), Notch Receptor 3 (NOTCH3; upregulated), Decoy Receptor 2 (DC2; TRAIL4; upregulated) or a combination of any of the above. As would be appreciated by the skilled artisan, mRNA expression levels can be measured using any technique known in the art, including, but not limited to RNA sequencing, quantitative PCR, RT-qPCR, or a combination of any of the above. As would be appreciated by the skilled artisan, protein expression levels can be measured using any technique known in the art, including, but not limited to, fluorescence flow cytometry, western blotting, or any combination of any of the above. In some embodiments, cellular senescence is identified by methods known in the art, including, but not limited to BrdU staining assays, EdU staining assays, SA-β-galactosidase staining assays, SA-α-Fucosidase staining assays, lipofuscin staining assays, or any combination of any of the above.

Adverse Event (AE) means any untoward medical occurrence in a participant to whom a medicinal product has been administered, including occurrences which are not necessarily caused by or related to that product.

Serious Adverse Event (SAE) means any untoward medical occurrence that: results in death; is life-threatening; requires inpatient hospitalization or prolongation of existing hospitalization; results in persistent or significant disability/incapacity; consists of a congenital anomaly or birth defect; other important medical events that jeopardize the participant or require intervention to prevent one of the above consequences

Life-threatening refers to an event in which the participant was at immediate risk of death at the time of the event; it does not refer to an event which hypothetically might have caused death if it were more severe.

Adverse Reaction (AR) means an untoward or unintended response in a participant to an investigational medicinal product which is related to any dose administered to that participant.

The phrase “response to an investigational medicinal product” means that a causal relationship between a trial medication and an AE is at least a reasonable possibility i.e. the relationship cannot be ruled out. Cognitive disorders (CDs), also known as neurocognitive disorders (NCDs), are a category of mental health disorders that primarily affect cognitive abilities including learning, memory, perception, and problem solving. Neurocognitive disorders include delirium and mild and major neurocognitive disorder (previously known as dementia). They are defined by deficits in cognitive ability that are acquired (as opposed to developmental), typically represent decline, and may have an underlying brain pathology. The Diagnostic and Statistical Manual of Mental Disorders, Fifth Edition (DSM-5) defines six key domains of cognitive function: executive function, learning and memory, perceptual-motor function, language, complex attention, and social cognition.

Cognitive (neurocognitive) diseases and disorders include, for example, Alzheimer's disease, dementia, Huntington's disease, Lewy body disease (encompassing dementia with Lewy bodies and Parkinson's disease (PD)), traumatic brain injury (TBI), prion disease, and dementia/neurocognitive issues due to HIV infection.

Neurocognitive disorders can have numerous causes: genetics, brain trauma, stroke, and heart issues. The main causes are neurodegenerative diseases such as Alzheimer's disease dementia, Parkinson's disease, and Huntington's disease because they cause progressive neuronal loss. Other diseases and conditions that cause NDCs include vascular dementia, frontotemporal degeneration, Lewy body disease, prion disease, normal pressure hydrocephalus, and dementia/neurocognitive issues due to HIV infection. They may also include dementia due to substance abuse or exposure to toxins. Neurocognitive disorders may also be caused by brain trauma, including concussions and traumatic brain injuries, as well as post-traumatic stress and alcoholism. This is referred to as amnesia and is characterized by damage to major memory encoding parts of the brain such as the hippocampus. Difficulty creating recent term memories is called anterograde amnesia and is caused by damage to the hippocampus part of the brain, which is a major part of the memory process. Retrograde amnesia is also caused by damage to the hippocampus, but the memories that were encoded or in the process of being encoded in long term memory are erased.

Neurodegenerative diseases, disorders, or conditions include, but are not limited to, Alzheimer's disease (AD) and other dementias, Parkinson's disease (PD) and PD-related disorders (e.g., Prion disease, Motor neuron diseases (MND), Huntington's disease (HD), Spinocerebellar ataxia (SCA), and Spinal muscular atrophy (SMA)).

Neurological diseases, disorders, or conditions include but are not limited to amyotrophic lateral sclerosis (ALS), arteriovenous malformation, brain aneurysm, brain tumors, dural arteriovenous fistulae, epilepsy, headache, memory disorders, multiple sclerosis, Parkinson's disease, peripheral neuropathy, post-herpetic neuralgia, spinal cord tumor, and stroke.

The term “Idiopathic Parkinson's Disease” (IPD) or “Parkinson's disease” (PD), as used herein, refers to a common neurodegenerative disorder causing a wide spectrum of motor and nonmotor symptoms. The cardinal motor features include resting tremor, bradykinesia, rigidity, and at a later stage, postural instability. The diagnosis is clinical, and ancillary laboratory or radiology tests are unnecessary in typical cases. Despite the use of validated diagnostic criteria, misdiagnosis is relatively common, especially early in the disease process. This is largely due to the phenotypic heterogeneity in the idiopathic Parkinson's disease population as well phenotypic overlapping with other diseases. The diseases most commonly confused with idiopathic Parkinson's disease are the atypical parkinsonian disorders (dementia with Lewy bodies, multiple system atrophy (MSA), progressive supranuclear palsy (PSP), and corticobasal degeneration (CBD)), vascular parkinsonism, drug-induced parkinsonism, dopa responsive dystonia, normal pressure hydrocephalus, and essential tremor. Since the diagnosis of many of these other diseases is also clinical, familiarity with their typical presentations and most current diagnostic criteria is helpful. Brain MRI can be helpful in diagnosing some of the diseases, though brain imaging is most commonly unremarkable in idiopathic Parkinson's disease. DaTscan has an FDA indication to assist in the evaluation of adults with parkinsonian syndromes. It should not be used in typical cases but can be a useful adjunct especially in the differentiation of PD versus essential tremor or drug-induced parkinsonism (where the DaTscan should be normal).

For clinicians, distinguishing early IPD from other parkinsonian syndromes can be extraordinarily challenging because these conditions, especially in the earliest stages, are present with highly variable yet overlapping phenotypes. Most of the diseases in the differential diagnosis, including IPD itself, are clinical diagnoses made on the basis of history and examination without the benefit of laboratory or radiology data. Early and accurate diagnosis is of paramount importance for many reasons. First, treatment approaches differ significantly across many of these diseases. Second, as neuroprotective interventions that are currently under investigation become available, long-term outcomes may significantly improve with earlier diagnosis and intervention. Third, some of these diseases are prognostically very different from one another, so accurate diagnosis enables better counseling and setting realistic expectations for progression.

Idiopathic Parkinson's disease is characterized by reduced nigrostriatal and cortical dopaminergic influence, with changes in movement and can lead to behavioral and cognitive disturbances in many cases. Cognitive disturbances can occur during any stage of PD, but become more common as the disease progresses. Changes range from mild cognitive impairment in PD (PD-MCI) to PD dementia (PDD). Patients with PD-related cognitive impairment (CI) experience reduced ability to work, diminished quality of life, and greater mortality. A wide variety of neuropsychological tests have been used to evaluate cognitive decline.

PD dementia (PDD) has a unique clinical profile, distinct from Alzheimer's disease (AD) in the early stages, although neuropathology can overlap in both diseases. Cholinergic deficits, a feature of both AD and PDD, underlie the rationale for cholinesterase inhibitor therapy in both conditions. In clinical practice, it is important that PDD should be recognized and appropriately treated. Although the cholinesterase inhibitor rivastigmine has recently been approved for the management of PDD, there remains a need for the development of novel therapies that can affect key mechanisms of the disease or prevent/delay patients with PD and mild cognitive impairment from progressing to PDD. (Poewe et al., Int J Clin Pract. (2008) 62(10): 1581-1587).

Dementia is highly frequent in Parkinson' disease (PD) and has a major impact on different markers of quality of life. Heterogeneous cognitive dysfunction of a lesser severity is also common in newly diagnosed and prevalent PD patients without dementia. This mild cognitive impairment of PD (PD-MCI) is considered to confer a high risk to evolve to dementia. MCI is a transitional state between normality and dementia, with subjective or objective evidence of cognitive impairment, although activities of daily living are not affected. Major formal differentiation of PD-MCI from dementia has typically required cognitive deficits not to interfere significantly with the patient's ability to implement instrumental activities of daily living (IADL). While a certain degree of functional impairment can be identified in subjects diagnosed with PD-MCI when accurately measured, little is known on the impact of cognitive impairment on IADL in the pre-dementia stages of PD. (J. Kulisevsky et al. Parkinsonism and Related Disorders (2013) 19: 812-817)

Decreases in semantic fluency can suggest more posteriorly-mediated cognitive dysfunction among individuals with Parkinson's disease (PD) (Williams-Gray, Caroline H et al. “The distinct cognitive syndromes of Parkinson's disease: 5 year follow-up of the CamPaIGN cohort.” Brain: a journal of neurology vol. 132, Pt 11 (2009): 2958-69). Clinicians monitor verbal fluency scores to evaluate cognitive decline among individuals with PD. Although PD patients are known to suffer from difficulties with both phonemic and semantic verbal fluency, the latter is thought to be a poorer prognosis due to differing underlying mechanisms.

HADS: The Hospital Anxiety and Depression Scale (HADS) is a 14 items measurement tool to assess anxiety and depression. Each item scores from 0 to 3. Seven of the items relate to anxiety domain, and the other seven items are for depression domain. Scores of eleven or greater than eleven on each symptom domain facilitate the identification of the symptom.

ESS: The Epworth Sleep Scale is a short questionnaire to measure daytime sleepiness. It includes 8 questions with each answer rated from 0 to 3. Possible results range from 0 to 24. Scores below 9 are considered to be in normal range while scores equal to and more than 9 may need specialist assessment for further investigation and treatment.

FACIT-F: The Functional Assessment of Chronic Illness Therapy-Fatigue (FACIT-F) scale was developed from interviews with oncology patients and clinical experts to assess anemia-associated fatigue. It is a patient-reported rating scale consisting of 13 items (statements) with five ordered response categories (“not at all”-“a little bit”-“somewhat”-“quite a bit”-“very much”) regarding the respondents' situation during the past week. It yields a summed total score ranging between 0 and 52 (52=less fatigue). Item contents cover the experience (e.g., feelings of tiredness, listlessness, energy) as well as the impact (e.g., trouble doing things, need to sleep, social limitations) of fatigue but does not provide a definition of fatigue. It has been used and validated in a range of patient groups, such as rheumatoid arthritis, PD, various forms of cancer, and in the general population. The Pittsburgh Sleep Scale/Pittsburgh Sleep Quality Index is a self-reported questionnaire to evaluate the quality of sleep over one month period. It includes 19 items which are used to create seven components (subjective sleep quality, sleep latency, sleep duration, habitual sleep efficiency, sleep disturbances, use of sleeping medication and Daytime dysfunction). Each component has score 0-3 with possible score ranging from 0-21 with lower scores denoting a healthier sleep quality.

EQ-5D is a 5 item, validated general quality of life measure form which health utility can be calculated. This is a score within the range 0-1.0 for quality of life, where 0 is equivalent to death and 1 represents perfect health.

PDQ-39: The Parkinson's Disease Questionnaire (PDQ-39) (Peto V, Jenkinson C, Fitzpatrick R, Greenhall R. The development and validation of a short measure of functioning and well being for individuals with Parkinson's disease. Qual Life Res. 1995; 4(3):241-8) is used as a measure of global quality of life (QoL). This is a well validated tool with scores ranging from 0 (best QoL) to 100 (worst possible QoL score). Comprised of 39 items, it assesses health related quality of life (HRQoL) across eight domains encompassing mobility (10 items), ADLs (six items), emotional well-being (six items), stigma (four items), social support (three items), cognition (four items), communication (three items), and bodily discomfort (three items). Responses are recorded on a scale ranging from 0 (never) to 4 (always). The score from each subscale is transformed into a 0 to 100 score by summing the raw score from each item, dividing by the maximum possible raw score, and multiplying by 100. A composite summary index score (PDQ-SI) of the eight domains provides an overall measure of the impact of illness on functioning and well-being that reduces the role of chance, which may occur when exploring individual PDQ-39 domains. This is calculated using the mean of each domain score: Higher scores indicate poorer perceived health.

CANTAB: The Cambridge Neuropsychological Test Automated Battery (CANTAB) is a computer-based cognitive assessment system. The CANTAB is a validated, automated, neuropsychological battery, with vast possibilities to assess various cognitive functions, such as attention, learning, memory, problem solving, and executive function (Robbins T W, et al. Cambridge Neuropsychological Test Automated Battery (CANTAB): a factor analytic study of a large sample of normal elderly volunteers. Dementia. 1994; 5:266-81).

Structural (3D T1w) imaging is used to evaluate disease-related differences in brain macrostructure, particularly in grey matter. In addition to voxel wise statistical comparison of changes in grey matter, these scans can also be specifically analyzed for changes in hippocampal volume as have already been reported in PBC by the Swain group.

T2w FLAIR imaging refers to adding to the structural information to identify any areas of gross white matter disease via quantification of white matter hyperintensities (WMH) which have also been associated with PBC.

Diffusion Tensor Imaging (DTI) is used for tract based spatial statistical (TBSS) analysis of white matter tract changes, building on the highly significant correlation between white matter fractional anisotropy and functional measures in pilot PBC studies.

T1 mapping is another exploratory approach which gives quantitative values on tissue properties and is used to detect subtle reversible changes in the grey matter.

Resting state functional MRI scan measures interconnection of cortical brain areas which form networks based on correlated slowly varying brain activity.

Proton magnetic resonance spectroscopy (Proton MRS) is a noninvasive neuroimaging technique that quantitatively measures metabolite changes (Murray, Melissa E et al. “Early Alzheimer's disease neuropathology detected by proton MR spectroscopy.” The Journal of neuroscience: the official journal of the Society for Neuroscience vol. 34, 49 (2014): 16247-55). Proton MRS measurements of the metabolite N-acetylaspartate (NAA) and the ratio of NAA to the reference metabolite creatine (NAA/Cr) longitudinally decrease over the course of AD dementia and mild cognitive impairment.

MDS-UPDRS: The Movement Disorder Society revised Unified Parkinson's Disease Rating Scale (MDS-UPDRS) Part III is based on examination of the participant and is a measure of motor severity (range 0-132). Each parkinsonian sign is rated on a 5-point Likert scale (ranging from 0 to 4), with higher scores indicating a more severe impairment. This should be performed whilst the participant is “on”—the typical functioning state when patients are receiving medication and have a good response.

The Hoehn and Yahr Stage is a long standing internationally established scale that mirrors disability of patients and is as below:
Stage 0: No signs of disease
Stage 1: Unilateral disease
Stage 2: Bilateral disease, without impairment of balance
Stage 3: Mild to moderate bilateral disease; some postural instability and needs assistance to recover from the pull test; physically independent
Stage 4: Severe disability; still able to walk or stand unassisted
Stage 5: Wheelchair bound or bedridden unless aided.

PD-CFRS: The Parkinson's Disease Cognitive Functional Rating Scale (PD-CFRS) is a five minute PD-specific questionnaire which explores a wide range of functional aspects suspected to be sensitive to cognitive impairment in PD, minimising the motor impact of the disease. It measures the functional impact of cognitive impairment in PD and change over time. It can be repeated after six months, at which time an increase of two points indicates a clinically significant worsening of the cognitive functional status. It is not subject to confounding by motor disability, age, education, anxiety, or depression. It is valid, reliable, and offers a reasonable sensitivity of 84% and specificity of 69% at a cut-off of greater than two to detect PD with mild cognitive impairment (PD-MCI) with associated functional impairment. It can also diagnose PD dementia (PDD) with a sensitivity and specificity of 83% using a higher cut-off value of greater than six.

The scale is administered to a knowledgeable informant in interview form by 12 items selected to cover the spectrum of instrumental cognitive changes seen in PD over the last two weeks before the evaluation. All 12 questions explore with some examples, whether or not the patient has had trouble in performing an activity (0=none; 1=some of the time; 2=most of the time; 8=the subject has never done the activity in the past) such as handling money, domestic economy, arranging holidays or meetings, handling personal mail, controlling drug treatment schedule, organizing daily activities, handling home electrical appliances, understanding how to use public transport, solving unforeseen events, explaining things he/she want to say, understanding the things he/she read, and handling the cell phone. The maximum score, obtained by the sum of the ratings, is 24. Whenever there are “eights” in the record sheet indicating an activity never done in the past, the mean of all the items answered 0-2 is calculated, and this mean value (either 0, 1 or 2) is scored in those items answered with an eight (J. Kulisevsky et al. Parkinsonism and Related Disorders (2013) 19: 812-817).

MOCA: The Montreal Cognitive Assessment (MOCA) is a measure of global cognitive function (maximum 30). Scores <26 may indicate a degree of impaired cognition.

Farnesoid X receptor (FXR), a metabolic nuclear receptor, plays critical roles in the maintenance of systemic energy homeostasis and the integrity of many organs, including liver and intestine. It regulates bile acid, lipid, and glucose metabolism, and contributes to inter-organ communication, in particular the enterohepatic signaling pathway, through bile acids and fibroblast growth factor-15/19 (FGF-15/19). The metabolic effects of FXR are also involved in gut microbiota. FXR has various functions in the kidney, adipose tissue, pancreas, cardiovascular system, and tumorigenesis. The deregulation of FXR may lead to abnormalities of specific organs and metabolic dysfunction, allowing the protein as an attractive therapeutic target for the management of liver and/or metabolic diseases.

A “pharmaceutical composition” is a formulation containing a compound of formula (I) (e.g., a compound of formula (A), (B), (C), or (D)) in a form suitable for administration to a subject. Pharmaceutical composition can contain at least one pharmaceutically acceptable excipient.

As used herein, the term “pharmaceutically acceptable excipient” refers to an excipient that is useful in preparing a pharmaceutical composition that is generally safe, non-toxic, and neither biologically nor otherwise undesirable, and includes excipient that is acceptable for veterinary use as well as human pharmaceutical use.

A “composition of a compound of formula (I)” described herein refers to a compound of formula (I) administered to a patient in any form described herein including as a component of a pharmaceutical composition. An “obeticholic acid composition” described herein refers to obeticholic acid administered to a patient in any form described herein including as a component of a pharmaceutical composition.

The term “effective amount” as used herein refers to an amount of a compound of formula (I) (e.g., a compound of formula (A), (B), (C), or (D)) (e.g., an FXR-activating ligand) that produces an acute or chronic therapeutic effect upon appropriate dose administration. In some aspects the present disclosure provides a method for treating a disease or condition by administering an effective amount of an active compound or pharmaceutical composition described herein (e.g., OCA or OCA composition) to a patient in need thereof.

“A therapeutically effective amount” means, for example, the amount of a compound of formula (I) (e.g., a compound of formula (A), (B), (C), or (D)) that, when administered to a mammal for treating a disease, is sufficient to affect such treatment for the disease. The “therapeutically effective amount” will vary depending on the active agent (e.g., obeticholic acid), the disease and its severity and the age, weight, etc., of the mammal to be treated. A therapeutically effective amount can refer to a starting dose or adjusted dose as set forth herein.

A therapeutically effective amount of an active compound as described herein (e.g., obeticholic acid) can be formulated with a pharmaceutically acceptable carrier for administration to a human or an animal. Accordingly, an active compound as described herein (e.g., obeticholic acid) or its formulations can be administered, for example, via oral, parenteral, or topical routes, to provide an effective amount of the compound.

The term “regimen” refers to a protocol for dosing and/or timing the administration of one or more therapies (e.g., a composition of a compound of formula (I) (e.g., a compound of formula (A), (B), (C), or (D)) described herein for treating a disease, disorder, or condition described herein). A regimen can include periods of active administration and periods of rest as known in the art. Active administration periods include administration of the compositions of a compound of formula (I) (e.g., a compound of formula (A), (B), (C), or (D)) described herein in a defined course of time, including, for example, the number of and timing of dosages of the compositions. In some regimens, one or more rest periods can be included where no compound is actively administered, and in certain instances, includes time periods where the efficacy of such compounds can be minimal.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

The term “administering” refers to the act of delivering a composition of a compound of formula (I) (e.g., a compound of formula (A), (B), (C), or (D)) described herein into a subject by such routes as oral, mucosal, topical, suppository, intravenous, parenteral, intraperitoneal, intramuscular, intralesional, intrathecal, intranasal or subcutaneous administration. The term can also refer to the frequency (e.g., daily, weekly, monthly, etc.) of providing a composition of a compound of formula (I) (e.g., a compound of formula (A), (B), (C), or (D)) described herein to a patient. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition). In certain embodiments, administration as used herein refers to oral administration.

“Pharmacological effect” as used herein encompasses effects produced in the subject that achieve the intended purpose of a therapy. In one embodiment, a pharmacological effect means that primary indications of the subject being treated are prevented, alleviated, or reduced. For example, a pharmacological effect would be one that results in the prevention, alleviation, or reduction of primary indications in a treated subject. In some embodiments, a pharmacological effect means that disorders or symptoms of the primary indications of the subject being treated are prevented, alleviated, or reduced. For example, a pharmacological effect would be one that results in the prevention or reduction of primary indications in a treated subject.

The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient as described above.

The unit dosage form is any of a variety of forms, including, for example, a capsule, an IV bag, a tablet, a single pump on an aerosol inhaler, or a vial. The quantity of a compound of formula (I) (e.g., a compound of formula (A), (B), (C), or (D)) (e.g., a formulation of a compound of formula (I) (e.g., a compound of formula (A), (B), (C), or (D)), or a pharmaceutically acceptable salt, ester, solvate, or amino acid conjugate thereof) in a unit dose of composition is an effective amount and is varied according to the particular treatment involved. One skilled in the art will appreciate that it is sometimes necessary to make routine variations to the dosage, depending on the age and condition of the patient. The dosage will also depend on the route of administration (e.g., oral, pulmonary, rectal, parenteral, transdermal, subcutaneous, intravenous, intramuscular, intraperitoneal, inhalational, buccal, sublingual, intrapleural, intrathecal, intranasal, and the like).

The term “flash dose” refers to formulations of a compound of formula (I) (e.g., a compound of formula (A), (B), (C), or (D)) that are rapidly dispersing dosage forms.

The term “immediate release” is defined as a release of a compound of formula (I) (e.g., a compound of formula (A), (B), (C), or (D)) from a dosage form in a relatively brief period of time, generally up to about 60 minutes.

A “subject” or “patient” includes mammals, e.g., humans, companion animals (e.g., dogs, cats, birds, and the like), farm animals (e.g., cows, sheep, pigs, horses, fowl, and the like), and laboratory animals (e.g., rats, mice, guinea pigs, birds, and the like). In one embodiment, the patient is human. In one embodiment, the subject is not suffering from a cholestatic condition. Patients described herein include patients having a disease or condition described herein. A patient can be described or referred to by the condition treated. For example, a patient having PD can be referred to herein as a PD patient. A patient described herein can have a preexisting condition (e.g., a condition other than the disease or condition treated by a compound of formula (I) (e.g., a compound of formula (A), (B), (C), or (D)) or composition described herein that existed at the time of first administration). In one instance a patient described is an elderly/geriatric patient.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A “control” as used herein refers to a baseline level determined on a patient-by-patient basis (or animal if in animal models), an amount or level considered by those skilled in the art as a normal value, or any level or measure of a condition or biomarker described herein taken from a patient or population of patients at any given time for a given condition.

The present application relates to a method of ameliorating, mitigating, treating, or preventing cognitive impairment in a subject comprising administering to the subject at least one therapeutically effective amount of a compound of formula (I):

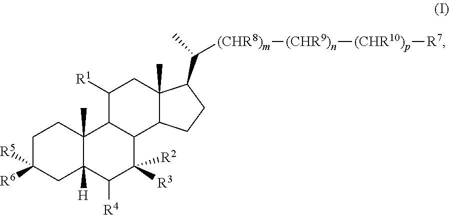

or a pharmaceutically acceptable salt, ester, solvate, or amino acid conjugate (such as, e.g., glycine, taurine or sarcosine conjugate) thereof, wherein:
R¹ is H, OH, alkoxy, or oxo;
R² is H, OH, halogen, or alkyl optionally substituted with one or more halogen or OH, or R² and R³ taken together with the carbon atom to which they are attached form a carbonyl;
R³ is H, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, or halogen, or R⁵ and R⁶ taken together with the carbon atom to which they are attached form a carbonyl;
R⁷ is OH, OSO₃H, SO₃H, OSO₂NH₂, SO₂NH₂, OPO₃H₂, PO₃H₂, CO₂H, C(O)NHOH, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, or 2,4-difluoro-3-hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
m is 0, 1, or 2;
n is 0 or 1; and
p is 0 or 1.

In some embodiments, the present application relates to the use of a compound of formula (I), or a pharmaceutically acceptable salt, ester, solvate, or amino acid conjugate (such as, e.g., glycine, taurine or sarcosine conjugate) thereof, for ameliorating, mitigating, treating, or preventing cognitive diseases, disorders, or conditions.

In some embodiments, the present application relates to the use of a compound of formula (I), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing cognitive diseases, disorders, or conditions.

In some embodiments, the present application relates to a compound of formula (I), or a pharmaceutically acceptable salt, ester, solvate, or amino acid conjugate (such as, e.g., glycine, taurine or sarcosine conjugate) thereof, for use in ameliorating, mitigating, treating, or preventing cognitive diseases, disorders, or conditions.

In some embodiments, the present application relates to a kit for ameliorating, mitigating, treating, or preventing cognitive diseases, disorders, or conditions, comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing cognitive diseases, disorders, or conditions, comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

In some embodiments, the compound of formula (I) is a compound of formula (A)

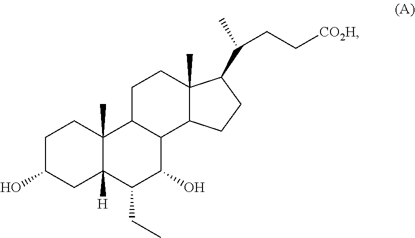 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or a pharmaceutically acceptable salt, ester, solvate, or amino acid conjugate (such as, e.g., glycine, taurine or sarcosine conjugate) thereof.

In some embodiments, the compound of formula (I) is a compound of formula (B):

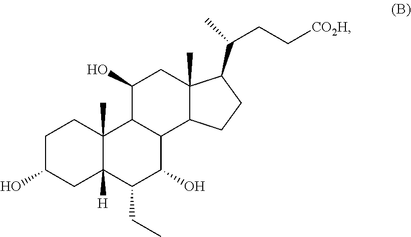 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

or a pharmaceutically acceptable salt, ester, solvate, or amino acid conjugate (such as, e.g., glycine, taurine or sarcosine conjugate) thereof.

In some embodiments, the present application is directed to methods of ameliorating, mitigating, treating, or preventing cognitive diseases, disorders or conditions using a compound of formula (I) or a pharmaceutically acceptable salt, ester, solvate, or amino acid conjugate thereof, wherein the compound of formula (I) is a compound of formula (C):

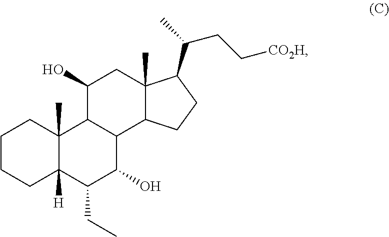 [SMILES: CC[C@H]1[C@@H](O)C2C3CC[C@H]([C@H](C)CCC(=O)O)[C@@]3(C)C[C@H](O)C2[C@@]2(C)CCCC[C@@H]12]

or a pharmaceutically acceptable salt, ester, solvate, or amino acid conjugate (such as, e.g., glycine, taurine or sarcosine conjugate) thereof.

In some embodiments, the compound of formula (I) is a compound of formula (D):

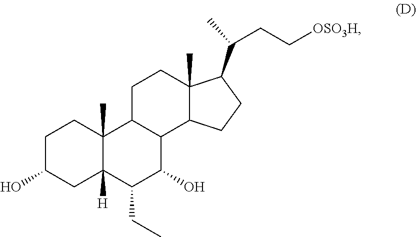

or pharmaceutically acceptable salt, ester, solvate, or amino acid conjugate (such as, e.g., glycine, taurine or sarcosine conjugate) thereof.

In certain embodiments, the compound of formula (I) (e.g., compound of formula (A), (B), (C), or (D)) is a free form (e.g., free acid). In certain embodiments, the compound of formula (I) (e.g., compound of formula (A), (B), (C), or (D)) is a pharmaceutically acceptable salt. In certain embodiments, the compound of formula (I) (e.g., compound of formula (A), (B), (C), or (D)) is a solvate. In certain embodiments the compound of formula (I) (e.g., compound of formula (A), (B), (C), or (D)) is an ester. In certain embodiments the compound of formula (I) (e.g., compound of formula (A), (B), or (C)) is an amino acid conjugate.

The present disclosure relates to a method of ameliorating, mitigating, treating, or preventing cognitive impairment associated with cognitive, neurocognitive, neurological, or neurodegenerative disease, disorder or condition (e.g., Parkinson's or Parkinson's-related disease, disorder or condition) described herein, in a subject in need thereof, comprising administering to the subject a therapeutically effective amount of the compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some embodiments, the cognitive impairment is associated with a neurological disease. In certain aspects of the disclosure, the cognitive impairment is associated with a neurodegenerative disease, disorder, or condition. In one of the aspects, the neurodegenerative disease is Parkinson's disease (PD). In certain embodiments of the present disclosure, the disease associated with cognitive impairment is Parkinson's disease. In certain embodiments of the present disclosure, the disease associated with cognitive impairment is Parkinson's disease dementia. In some embodiments, the disease, disorder or condition is cognitive impairment (CI) in PD dementia. In some embodiments, the disease, disorder or condition is mild cognitive impairment in PD (PD-MCI)).

In some aspects, the present disclosure relates to a method of ameliorating, mitigating, treating, or preventing a neurodegenerative disease or disorder (e.g., Parkinson's or Parkinson's-related disease, disorder or condition) described herein, in a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing a neurodegenerative disease or disorder (e.g., Parkinson's or Parkinson's-related disease, disorder or condition) described herein in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing a neurodegenerative disease or disorder (e.g., Parkinson's or Parkinson's-related disease, disorder or condition) described herein in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing a neurodegenerative disease or disorder (e.g., Parkinson's or Parkinson's-related disease, disorder or condition) described herein in a subject in need thereof.

In some embodiments, the present application relates to a kit for ameliorating, mitigating, treating, or preventing a neurodegenerative disease or disorder (e.g., Parkinson's or Parkinson's-related disease, disorder or condition) described herein comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing a neurodegenerative disease or disorder (e.g., Parkinson's or Parkinson's-related disease, disorder or condition) described herein comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

In some embodiments, the neurodegenerative disease or disorder is Alzheimer's disease (AD) and other dementias, Parkinson's disease (PD) and PD-related disorders, Prion disease, Motor neuron diseases (MND), Huntington's disease (HD), Spinocerebellar ataxia (SCA), or Spinal muscular atrophy (SMA). In some embodiments, the neurodegenerative disease or disorder is Parkinson's disease.

In some aspects, the present disclosure relates to a method of using a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof for ameliorating, mitigating, treating, or preventing a neurological disease (e.g., chronic neurological disease), such as, for example, Parkinson's disease.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing a neurological disease (e.g., chronic neurological disease), such as, for example, Parkinson's disease in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing a neurological disease (e.g., chronic neurological disease), such as, for example, Parkinson's disease in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing a neurological disease (e.g., chronic neurological disease), such as, for example, Parkinson's disease in a subject in need thereof.

In some embodiments, the present application relates to a kit for ameliorating, mitigating, treating, or preventing a neurological disease (e.g., chronic neurological disease), such as, for example, Parkinson's disease, comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing a neurological disease (e.g., chronic neurological disease), such as, for example, Parkinson's disease comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

In some embodiments, the neurological disease is amyotrophic lateral sclerosis (ALS), arteriovenous malformation, brain aneurysm, brain tumors, dural arteriovenous fistulae, epilepsy, headache, memory disorders, multiple sclerosis, Parkinson's disease, peripheral neuropathy, post-herpetic neuralgia, spinal cord tumor, or stroke. In some embodiments, the neurological disease is Parkinson's disease.

In some aspects, the present disclosure relates to a method of using a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof for ameliorating, mitigating, treating, or preventing a cognitive disease, disorder or condition.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing a cognitive disease, disorder or condition in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing a cognitive disease, disorder or condition in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing a cognitive disease, disorder or condition herein in a subject in need thereof.

In some embodiments, the present application relates to a kit for ameliorating, mitigating, treating, or preventing a cognitive disease, disorder or condition herein comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing a cognitive disease, disorder or condition herein comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

In some aspects, the present disclosure relates to a method of using a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof for ameliorating, mitigating, treating, or preventing a neurocognitive disease, disorder or condition.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing a neurocognitive disease, disorder or condition in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing a neurocognitive disease, disorder or condition in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing a neurocognitive disease, disorder or condition herein in a subject in need thereof.

In some embodiments, the present application relates to a kit for ameliorating, mitigating, treating, or preventing a neurocognitive disease, disorder or condition herein comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing a neurocognitive disease, disorder or condition herein comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

In some aspects, the present disclosure relates to a method of ameliorating, mitigating, treating, or preventing a symptom (e.g., cognitive symptom, for example, cognitive impairment, cognitive deficit, cognitive disfunction) of a neurological disease or disorder (e.g., Parkinson's or Parkinson's-related disease, disorder or condition) described herein, in a subject in need thereof, comprising administering to the subject a therapeutically effective amount of a compound of formula (I) (e.g., compound of formula (A), (B), or (C)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing a symptom (e.g., cognitive symptom, for example, cognitive impairment, cognitive deficit, cognitive disfunction) of a neurological disease or disorder (e.g., Parkinson's or Parkinson's-related disease, disorder or condition) described herein in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing a symptom (e.g., cognitive symptom, for example, cognitive impairment, cognitive deficit, cognitive disfunction) of a neurological disease or disorder (e.g., Parkinson's or Parkinson's-related disease, disorder or condition) described herein in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing a symptom (e.g., cognitive symptom, for example, cognitive impairment, cognitive deficit, cognitive disfunction) of a neurological disease or disorder (e.g., Parkinson's or Parkinson's-related disease, disorder or condition) described herein in a subject in need thereof.

In some embodiments, the present application relates to a kit for ameliorating, mitigating, treating, or preventing a symptom (e.g., cognitive symptom, for example, cognitive impairment, cognitive deficit, cognitive disfunction) of a neurological disease or disorder (e.g., Parkinson's or Parkinson's-related disease, disorder or condition) described herein comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing a symptom (e.g., cognitive symptom, for example, cognitive impairment, cognitive deficit, cognitive disfunction) of a neurological disease or disorder (e.g., Parkinson's or Parkinson's-related disease, disorder or condition) described herein comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Cognitive symptoms include, for example, apathy, anxiety, depression, distorted thinking; difficulty concentrating; difficulty planning activities, distractibility; forgetfulness; reduced reaction time; memory loss (e.g., loss of short-term or long-term memory); indecisiveness; disorientation; impaired judgment; word-finding difficulties; difficulty shifting between tasks; slurred speech, slowness of thought; delusions; and hallucinations.

In some embodiments, the symptom of a neurological disease or disorder is a symptom of PD. In some embodiments, the symptom of a neurological disease or disorder is a symptom of PD dementia (PDD).

Another aspect of the disclosure relates to a method of ameliorating, mitigating, treating, or preventing a symptom (e.g., cognitive impairment) of Parkinson's disease, in a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing a symptom (e.g., cognitive impairment) of Parkinson's disease in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing a symptom (e.g., cognitive impairment) of Parkinson's disease in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing a symptom (e.g., cognitive impairment) of Parkinson's disease in a subject in need thereof.

In some embodiments, the present application relates to a kit for ameliorating, mitigating, treating, or preventing a symptom (e.g., cognitive impairment) of Parkinson's disease comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing a symptom (e.g., cognitive impairment) of Parkinson's disease comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of ameliorating, mitigating, treating, or preventing a risk factor for the development of PDD, in a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

Risk factors for the development of PDD include advanced age, male sex, higher UPDRS III scores, hallucinations, REM sleep behavior disorder (RBD), smoking, specific genetics, symmetry of motor symptoms, postural instability gait difficult phenotype, mild cognitive impairment at baseline, and hypertension. (Xu Y, Yang J, Shang H. Meta-analysis of risk factors for Parkinson's disease dementia. Transl Neurodegener. 2016; 5:11). They can also include excessive daytime sleepiness, orthostatic hypotension, reduced educational level and gastrointestinal or urological symptoms, however, study findings are heterogeneous.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing a risk factor for the development of PDD in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing a risk factor for the development of PDD in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing a risk factor for the development of PDD in a subject in need thereof.

In some embodiments, the present application relates to a kit for ameliorating, mitigating, treating, or preventing a risk factor for the development of PDD comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing a risk factor for the development of PDD comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of ameliorating, mitigating, treating, or preventing a mild cognitive impairment in PD dementia (PD-MCI), in a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing PD-MCI in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing PD-MCI in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing PD-MCI in a subject in need thereof.

In some embodiments, the present application relates to a kit for ameliorating, mitigating, treating, or preventing PD-MCI comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing PD-MCI comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of ameliorating, mitigating, treating, or preventing Parkinson's disease or PD-related disorder or condition, in a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing Parkinson's disease or PD-related disorder or condition in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing Parkinson's disease or PD-related disorder or condition in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing Parkinson's disease or PD-related disorder or condition in a subject in need thereof.

In some embodiments, the present application relates to a kit for ameliorating, mitigating, treating, or preventing Parkinson's disease or PD-related disorder or condition comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing Parkinson's disease or PD-related disorder or condition comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of ameliorating, mitigating, treating, or preventing PD dementia (PDD), in a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for ameliorating, mitigating, treating, or preventing PDD in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for ameliorating, mitigating, treating, or preventing PDD in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in ameliorating, mitigating, treating, or preventing PDD in a subject in need thereof.

In some embodiments, the present application relates to a kit for ameliorating, mitigating, treating, or preventing PDD comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for ameliorating, mitigating, treating, or preventing PDD comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of slowing down or reversing the progression of Parkinson's disease, in a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for slowing down or reversing the progression of Parkinson's disease in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for slowing down or reversing the progression of Parkinson's disease in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in slowing down or reversing the progression of Parkinson's disease in a subject in need thereof.

In some embodiments, the present application relates to a kit for slowing down or reversing the progression of Parkinson's disease comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for slowing down or reversing the progression of Parkinson's disease comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of slowing down or reversing the progression of PDD, in a subject in need thereof, comprising administering to the subject a therapeutically effective amount of a compound of formula (I) (e.g., compound of formula (A), (B), or (C)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for slowing down or reversing the progression of PDD in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for slowing down or reversing the progression of PDD in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in slowing down or reversing the progression of PDD in a subject in need thereof.

In some embodiments, the present application relates to a kit for slowing down or reversing the progression of PDD comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for slowing down or reversing the progression of PDD comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of slowing down or reversing the progression of PD-MCI, in a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for slowing down or reversing the progression of PD-MCI in a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for slowing down or reversing the progression of PD-MCI in a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in slowing down or reversing the progression of PD-MCI in a subject in need thereof.

In some embodiments, the present application relates to a kit for slowing down or reversing the progression of PD-MCI comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for slowing down or reversing the progression of PD-MCI comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of reversing cellular senescence associated with Parkinson's disease in the neurons of a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for reversing cellular senescence associated with Parkinson's disease in the neurons of a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for reversing cellular senescence associated with Parkinson's disease in the neurons of a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in reversing cellular senescence associated with Parkinson's disease in the neurons of a subject in need thereof.

In some embodiments, the present application relates to a kit for reversing cellular senescence associated with Parkinson's disease in the neurons of a subject in need thereof comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for reversing cellular senescence associated with Parkinson's disease in the neurons of a subject in need thereof comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

Another aspect of the disclosure relates to a method of treating telomere disfunction associated with Parkinson's disease in the neurons of a subject in need thereof, comprising administering to the subject a therapeutically effective amount of a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for treating telomere disfunction associated with Parkinson's disease in the neurons of a subject in need thereof.

In some aspects, the present disclosure relates to the use of a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for the manufacture of a medicament for treating telomere disfunction associated with Parkinson's disease in the neurons of a subject in need thereof.

In some aspects, the present disclosure relates to a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof, for use in treating telomere disfunction associated with Parkinson's disease in the neurons of a subject in need thereof.

In some embodiments, the present application relates to a kit for treating telomere disfunction associated with Parkinson's disease in the neurons of a subject in need thereof comprising a compound of formula (I), or a pharmaceutically acceptable salt, ester, solvate, or amino acid conjugate (such as, e.g., glycine, taurine or sarcosine conjugate) thereof and instructions for use.

In some embodiments, the present application is directed to a pharmaceutical composition for treating telomere disfunction associated with Parkinson's disease in the neurons of a subject in need thereof comprising a compound of formula (I), or a pharmaceutically acceptable salt, ester, solvate, or amino acid conjugate (such as, e.g., glycine, taurine or sarcosine conjugate) thereof and at least one pharmaceutically acceptable excipient.

The compounds of the present disclosure may be useful in enhancing cognition in a patient with a neurological disease, disorder or condition associated with a cognition deficit or impairment, or neurodegenerative or neurocognitive disease. The disclosure also relates to a compound of formula (I) (e.g., a compound of formula (A), (B), (C), or (D) or pharmaceutically acceptable salts, solvates, esters, or amino conjugates thereof) or a pharmaceutical compositions thereof comprising the compound of formula (I) (e.g., a compound of formula (A), (B), (C), or (D) or pharmaceutically acceptable salts, solvates, esters, or amino conjugates thereof) as defined above, for enhancing cognition in a patient with neurological disease, disorder or condition associated with a cognition deficit or impairment, or neurodegenerative or neurocognitive disease. In one aspect, the present disclosure relates to the use of a compound of formula (I) (e.g., a compound of formula (A), (B), (C), or (D) or pharmaceutically acceptable salts, solvates, esters, or amino conjugates thereof) as defined above, for the preparation of a medicament for the treatment and/or prevention of neurological diseases, disorders or conditions accompanied by a cognition deficit or impairment, or neurodegenerative or neurocognitive diseases; and may also be formulated as a method for the treatment and/or prevention of neurological diseases, disorders or conditions associated with (e.g., accompanied by) cognition deficit or impairment, or neurodegenerative or neurocognitive diseases comprising administering a therapeutically effective amount of the previously defined compound of formula (I) and one or more pharmaceutical acceptable excipients or carriers, in a subject in need thereof.

In some embodiments, the neurodegenerative diseases are neurodegenerative diseases accompanied by a cognition deficit or impairment. In some embodiments, the neurodegenerative disease or neurological disorder accompanied by cognition deficit or impairment is selected from Alzheimer's disease, Parkinson's disease, Huntington's disease, vascular dementia (uncomplicated, with delirium, with delusions or with depressed mood), mild cognitive impairment and age-associated cognition impairment. In some embodiments, the disease is Parkinson's disease.

In some embodiments, the disorders or conditions (e.g., dementia) associated with mild to severe cognition impairment and dementing illnesses include, for example, senile dementia, Alzheimer's disease, Pick's disease, frontotemporal dementia, supranuclear palsy, dementia with Lewy bodies, vascular dementia, Huntington's disease, Parkinson's disease, multiple sclerosis, amyotrophic lateral sclerosis, dementia associated with Down's syndrome, depression, Wernicke-Korsakoff's syndrome, corticobasal degenerations, and prion disease.

In one embodiment, the disease or condition associated with cognitive impairment as described herein is FXR-mediated. FXR mRNA and protein expression were identified in mouse brain neurons and in mouse/human brain tissues (Huang, et al., Identification of functional farnesoid X receptors in brain neurons. FEBS Lett. 2016, 590, 3233-3242); FXR was predominantly localized in the nucleus in cultured neurons, whereas it was mainly found in the cytoplasm in the neurons in vivo. The activation of FXR augmented the SHP expression levels in cultured neurons and in brain tissues, suggesting the potential functional roles of FXR in brain neurons.

The pharmaceutical compositions as described herein can be administered to a patient in an amount of between about 1 mg to about 50 mg; about 1 mg to about 40 mg; about 1 mg to about 30 mg; about 10 mg to about 30 mg; about 15 mg to about 30 mg; about 20 mg to about 30 mg; about 25 mg to about 30 mg; about 1 mg to about 25 mg; about 10 mg to about 25 mg; about 15 mg to about 25 mg; about 20 mg to about 25 mg; about 1 mg to about 20 mg; about 1 mg to about 10 mg; about 1 mg to about 5 mg; or about 1 mg to about 3 mg. In one embodiment, the pharmaceutical compositions as described herein can be administered to a patient in an amount of about 1, 1.5, 2, 2.5, 3, 4, 5, 6, 7, 8, 9, 10, 11, 12, 13, 14, 15, 16, 17, 18, 19, 20, 21, 22, 23, 24, 25, 30, 35, 40, 45, or 50 mg. In still other instances, the pharmaceutical compositions as described herein can be administered at an amount of about 1 mg, about 1.5 mg, about 3 mg, about 5 mg, about 10 mg, about 15 mg, about 25 mg, or about 50 mg. For example, an effective amount of an obeticholic acid composition described herein can be 1 mg, 1.5 mg, 3 mg, 5 mg, 10 mg, 25 mg, or 50 mg.

It is to be understood that, for example, the amount of a composition of a compound of formula (I) (e.g., compound of formula (A), (B), (C), or (D)) described herein as administered to a patient described herein refers to the amount of a compound of formula (I) (e.g., compound of formula (A), (B), (C), or (D)) in the composition. For example, the amount of an obeticholic acid composition described herein refers to the amount of obeticholic acid in the composition.

In certain embodiments, the amount of an obeticholic acid composition as provided above can refer to an effective amount as described herein. In certain embodiments, an effective amount of the obeticholic acid composition administered to a patient described herein can be 5 mg. In some embodiments, an effective amount of the obeticholic acid composition administered to a patient described herein can be 10 mg. In some embodiments, an effective amount of the obeticholic acid composition administered to a patient described herein can be 25 mg. In some embodiments, an effective amount of the obeticholic acid composition administered to a patient described herein can be 30 mg. In some embodiments, an effective amount of the obeticholic acid composition administered to a patient described herein can be 50 mg. In certain embodiments, an effective amount of the obeticholic acid composition administered to a patient described herein can be about 1 mg to about 50 mg; about 1 mg to about 40 mg; about 1 mg to about 30 mg; about 10 mg to about 30 mg; about 15 mg to about 30 mg; about 20 mg to about 30 mg; about 25 mg to about 30 mg; about 1 mg to about 25 mg; about 10 mg to about 25 mg; about 15 mg to about 25 mg; about 20 mg to about 25 mg; about 1 mg to about 20 mg; about 1 mg to about 10 mg; or about 1 mg to about 5 mg.

A pharmaceutical composition of the disclosure is formulated to be compatible with its intended route of administration. Examples of routes of administration include parenteral (e.g., intravenous, intradermal, subcutaneous), oral (e.g., inhalation), transdermal (topical), and transmucosal administration.

It can be useful to orally administer the pharmaceutical compositions described herein. Oral compositions generally include an inert diluent or an edible pharmaceutically acceptable carrier. They can be enclosed in gelatin capsules or compressed into tablets. For the purpose of oral therapeutic administration, the active compound (a compound of formula (I) (e.g., compound of formula (A), (B), (C), or (D)) as described herein, or a pharmaceutically acceptable salt, ester, solvate, or amino acid conjugate (such as, e.g., glycine, taurine or sarcosine conjugate) thereof (e.g., obeticholic acid)) or its particles, can be incorporated with excipients and used in the form of tablets, troches, or capsules.

The tablets, pills, capsules, troches and the like can contain any of the following ingredients, or compounds of a similar nature: a binder such as microcrystalline cellulose, gum tragacanth or gelatin; an excipient such as sodium starch glycolate, starch or lactose, a diluent such as microcrystalline cellulose, a disintegrating agent such as alginic acid, PRIMOJEL®, or corn starch; a lubricant such as magnesium stearate daily; a glidant such as colloidal silicon dioxide; a sweetening agent such as sucrose or saccharin; or a flavoring agent such as peppermint, methyl salicylate, or orange flavoring.

In one embodiment, the pharmaceutical composition further comprises one or more pharmaceutical excipients. The excipient can be any excipient present in the composition comprising a compound of formula (I) (e.g., compound of formula (A), (B), (C), or (D)), or a pharmaceutically acceptable salt, ester, solvate, or amino acid conjugate (such as, e.g., glycine, taurine or sarcosine conjugate) thereof (e.g., obeticholic acid). In one embodiment, the pharmaceutical composition comprises a therapeutically effective amount of obeticholic acid, or a pharmaceutically acceptable salt, ester, solvate, or amino acid conjugate (such as, e.g., glycine, taurine or sarcosine conjugate) thereof, and a pharmaceutically acceptable excipient.

The excipient can be one or more selected from the group consisting of diluents, sweeteners, viscosity enhancing agents, dispersing agents, preservatives, flavoring agents and the like. One excipient can perform more than one function. In one embodiment, the one or more pharmaceutical excipients include a lubricant and/or a diluent. In one embodiment, the excipient can be any excipient known in the art. Examples of excipients include, but are not limited to, calcium phosphate, microcrystalline cellulose, sodium starch glycolate and magnesium stearate, or a combination thereof. In some embodiments, the excipient is selected from calcium phosphate, microcrystalline cellulose, sodium starch glycolate and magnesium stearate. In some embodiments, the excipient is selected from microcrystalline cellulose, sodium starch glycolate and magnesium stearate. In some embodiments, the excipient is the excipient is magnesium stearate. In some embodiments, the excipient is microcrystalline cellulose. In a further embodiment, the excipient is sodium starch glycolate.

In one embodiment, the lubricant can be any lubricant known in the art. Non-limiting examples of lubricants include magnesium stearate, calcium stearate, stearic acid, glyceryl behenate, hydrogenated vegetable oil, sodium stearyl fumarate, and glyceryl fumarate, and/or a combination thereof. In some embodiments, the lubricant is selected from magnesium stearate, calcium stearate, stearic acid, glyceryl behenate, hydrogenated vegetable oil, sodium stearyl fumarate, and glyceryl fumarate. In some embodiments, the lubricant is calcium stearate. In some embodiments, the lubricant is stearic acid. In further embodiment, the lubricant is magnesium stearate.

In one embodiment, the diluent can be any diluent known in the art. Non-limiting examples of diluents include starch, pregelatinized starch, microcrystalline cellulose, calcium carbonate, dibasic calcium phosphate, tribasic calcium phosphate, calcium phosphate, lactose, dextrose, fructose, lactitol, lactose, magnesium carbonate, magnesium oxide, maltitol, maltodextrin, maltose, simethicone, sodium chloride, talc, xylitol, sorbitol, mannitol, and sucrose, and/or a combination thereof. In some embodiments, the diluent is selected from starch, pregelatinized starch, microcrystalline cellulose, calcium phosphate, lactose, sorbitol, mannitol, and sucrose. In some embodiments, the diluent is calcium phosphate. In some embodiments, the diluent is mannitol. In further embodiment, the diluent is microcrystalline cellulose.

In one embodiment, the pharmaceutical composition may further comprise a coating agent such as sugar-based coating agents, water-soluble film coating agents, enteric coating agents, and delayed release coating agents or a coating composition comprising any combination thereof. In some embodiments, the coating agent can be any coating agent known in the art. Examples of coating agents include, but are not limited to, saccharose used alone or together with any of the agents such as talc, calcium carbonate, calcium phosphate, calcium sulphate, gelatin, gum arabic, polyvinylpyrrolidone, and pullulan or any combination thereof, cellulose derivatives such as hydroxypropyl cellulose, hydroxypropyl methyl cellulose, hydroxyethyl cellulose, methyl hydroxyethyl cellulose, and sodium carboxymethyl cellulose; synthetic polymers such as polyvinyl acetal diethyl amino acetate, aminoalkyl methacrylate copolymers, and polyvinylpyrrolidone; polysaccharides such as pullulan; hydroxypropyl methyl cellulose phthalate; hydroxypropyl methyl cellulose acetate succinate; carboxymethyl ethyl cellulose; cellulose acetate phthalate; acrylic acid derivatives such as methacrylic acid copolymer L, methacrylic acid copolymer LD and methacrylic acid copolymer S; natural substances such as shellac; titanium dioxide; polyvinyl alcohol (e.g., Opadry®); polyethylene glycol; talc; lecithin; and/or combinations thereof. In one embodiment, the coating agent is selected from hydroxypropyl cellulose, hydroxypropyl methyl cellulose, hydroxyethyl cellulose, methyl hydroxyethyl cellulose, sodium carboxymethyl cellulose, polyvinyl acetal diethyl amino acetate, polyvinyl alcohol, polyethylene glycol, and lecithin, or a combination thereof. In some embodiments, the coating agent is Opadry® II (e.g., Opadry® II green, white, yellow, etc.).

Some aspects of the present disclosure relate to a pharmaceutical composition or pharmaceutical compositions as described above which comprise microcrystalline cellulose, sodium starch glycolate, and magnesium stearate as excipients. Such a composition can be provided in a dosage form set forth herein, e.g., an oral dosage form such as a tablet or coated tablet. Thus, in certain instances, the pharmaceutical composition (e.g., obeticholic acid composition) useful in the methods is a tablet or coated tablet for oral administration. In one embodiment, the oral dosage form of the pharmaceutical composition described herein (e.g., obeticholic acid composition) includes a film coating that includes one or more excipients selected from polyvinyl alcohol (part hydrolyzed), titanium dioxide, macrogol (polyethylene glycol 3350), talc, and iron oxide.

In one embodiment, the pharmaceutically composition is in solid particle form. Any inert excipient that is commonly used as a carrier or diluent may be used in the pharmaceutical composition of the present disclosure, such as for example, a gum, a starch, a sugar, a cellulosic material, a glycolate, an acrylate, or mixtures thereof. In one embodiment, the filler/diluent is microcrystalline cellulose. The pharmaceutical composition may further comprise a disintegrating agent (e.g., sodium starch glycolate) and/or a lubricant (e.g., magnesium stearate). Also, the pharmaceutical composition may comprise one or more additives selected from a buffer, a surfactant, a solubilizing agent, a plasticizer, an emulsifier, a stabilizing agent, a viscosity increasing agent, a sweetener, a film forming agent, or any combination thereof.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of the active compound (e.g., obeticholic acid) or its particles calculated to produce the desired therapeutic effect in association with the required pharmaceutical carrier. The specification for the dosage unit forms of the disclosure is dictated by and directly dependent on the unique characteristics of the active compound (e.g., obeticholic acid) or its particles and the particular therapeutic effect to be achieved.

The compounds of the present disclosure (e.g., obeticholic acid) are preferably formulated in a unit dosage form, each dosage containing from about 1 mg to about 50 mg. In some embodiments, the formulation comprises about 3 mg to about 30 mg. In some embodiments, the formulation comprises about 3 mg to about 25 mg. In some embodiments, the formulation comprises about 3 mg to about 10 mg. In some embodiments, the formulation comprises about 3 mg to about 5 mg. In some embodiments, the formulation comprises about 5 mg to about 30 mg. In some embodiments, the formulation comprises about 5 mg to about 25 mg. In some embodiments, the formulation comprises about 5 mg to about 10 mg. In some embodiments, the formulation comprises about 10 mg to about 30 mg. In some embodiments, the formulation comprises about 10 mg to about 25 mg. In some embodiments, the formulation comprises about 15 mg to about 30 mg. In some embodiments, the formulation comprises about 15 mg to about 25 mg. In one embodiment, the formulation comprises about 1.5 mg. In one embodiment, the formulation comprises about 3 mg.

In some embodiments, the formulation comprises about 25 mg of obeticholic acid. In some embodiments, the formulation comprises about 10 mg. In one embodiment, the formulation comprises about 5 mg. In some embodiments, the formulation comprises about 3 mg. In some embodiments, the formulation comprises about 1.5 mg. In some embodiments, the formulation comprises about 1 mg.

The active ingredient as described herein (e.g., obeticholic acid) is generally effective over a wide dosage range. For example, dosages per day normally fall within the range of about 0.0001 to about 30 mg/kg of body weight. In the treatment of adult humans, the range of about 0.1 to about 15 mg/kg/day, in single or divided dose, is especially preferred.

However, it will be understood that the amount of the active compound (e.g., obeticholic acid) actually administered will be determined by a physician, in the light of the relevant circumstances, including the condition to be treated, the chosen route of administration, the form of the active compound (e.g., obeticholic acid) administered, the age, weight, and response of the individual patient, and the severity of the patient's symptoms, and therefore the above dosage ranges are not intended to limit the scope of the disclosure in any way. In some instances, dosage levels below the lower limit of the aforesaid range may be more than adequate, while in other cases still larger doses may be employed without causing any harmful side effect, provided that such larger doses are first divided into several smaller doses for administration throughout the day.

The pharmaceutical compositions described herein (e.g., obeticholic acid composition) can be administered in accordance with a dosing regimen. A dosing regimen refers to continual and intermittent administration of a pharmaceutical composition described herein at one or more of the amounts described herein. Thus, in certain instances, a dosing regimen can include administration of a pharmaceutical compositions described herein continually for any number of days, weeks, months, or years as set forth herein. In other instances, a dosing regimen can include administration of a pharmaceutical composition described herein intermittently, where, for example, the composition is administered for one period of time followed by a rest period or off period where the pharmaceutical composition is not administered.

The pharmaceutical compositions described herein (e.g., obeticholic acid composition) useful in the methods of treating and/or preventing the diseases, disorders or conditions described herein include administration of such compositions daily (QD), every other day (Q2D), once a week (QW), twice a week (BID), three times a week (TIW), once a month (QM), or twice a month (Q2M). In one embodiment, the pharmaceutical composition described herein (e.g., obeticholic acid composition) is administered QD. Thus, an effective amount of the pharmaceutical composition described herein (e.g., obeticholic acid composition) can be administered QD to treat a disease or condition described herein.

The embodiments described above include administration at an amount described above. For example, an obeticholic acid composition described herein can be administered in a frequency provided above in an amount of 1.5 mg, 3 mg, 5 mg, 10 mg, 25 mg, or 50 mg.

The pharmaceutical composition described herein (e.g., obeticholic acid composition) can be administered for any number of days, weeks, months, or years, including indefinitely, provided that the dosage remains efficacious for the patient and the patient tolerates the dosage (e.g., an adjusted or re-adjusted dose as described herein). In certain instances, the pharmaceutical composition described herein (e.g., obeticholic acid composition) is administered to a patient described herein until loss of efficacy, or until development of unacceptable toxicity or undesired adverse effects, such as, for example, those described herein. Daily dosing of the pharmaceutical composition described herein (e.g., obeticholic acid composition) can be dependent upon patient tolerance to the dosage, composition, or frequency of administration. For example, daily dosing can be administered to a patient described herein where the patient tolerates a daily dosage amount (e.g., 1.5 mg, 3 mg, 5 mg, 10 mg, 25 mg, or 50 mg). Alternatively, or additionally, the daily dosing can be modified to increase or reduce the amount of the pharmaceutical composition described herein (e.g., obeticholic acid composition) as provided above where the patient is tolerant or is intolerant to the dose, respectively. In certain embodiments, modification of the adjusted dose (or any re-adjusted dose) can be performed after the patient's liver function is assessed, monitored, or measured as described herein. In certain instances, the adjusted dose (or re-adjusted dose) is increased or maintained (e.g., equivalent to a starting dose) where the liver function is not impaired. In other instances, the adjusted dose (or re-adjusted dose) is decreased or maintained (e.g., equivalent to a starting dose) where the patient's liver function is impaired.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The disclosure having now been described by way of written description, those of skill in the art will recognize that the disclosure can be practiced in a variety of embodiments and that the foregoing description and examples below are for purposes of illustration and not limitation of the claims that follow.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended. It is to be further understood that resort may be had to various other embodiments, modifications, and equivalents thereof which may suggest themselves to those skilled in the art without departing from the spirit of the present disclosure and/or scope of the appended claims.

### Example 1: Non-Cholestatic Cognitive Decline

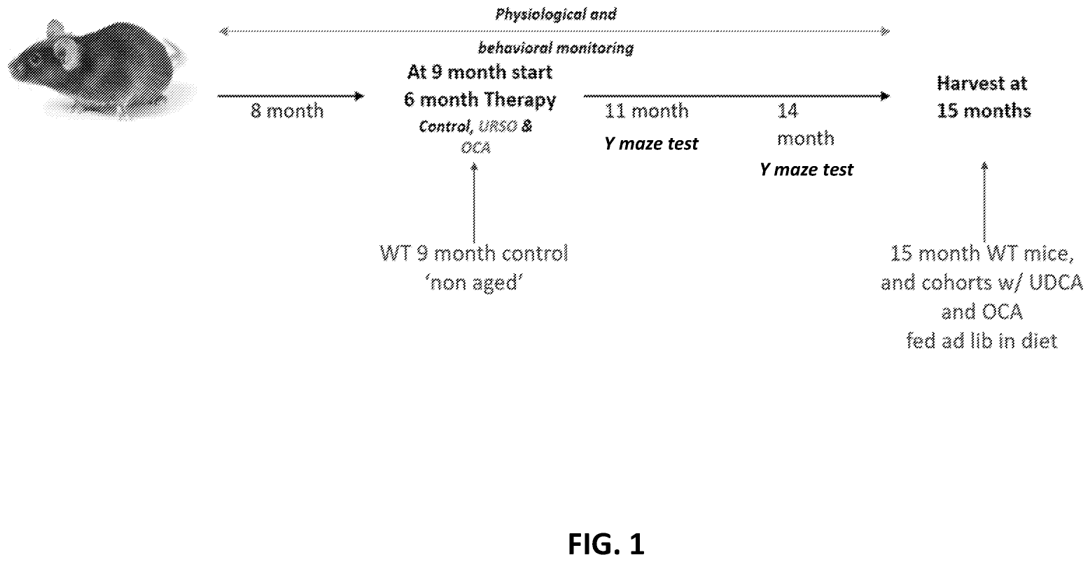

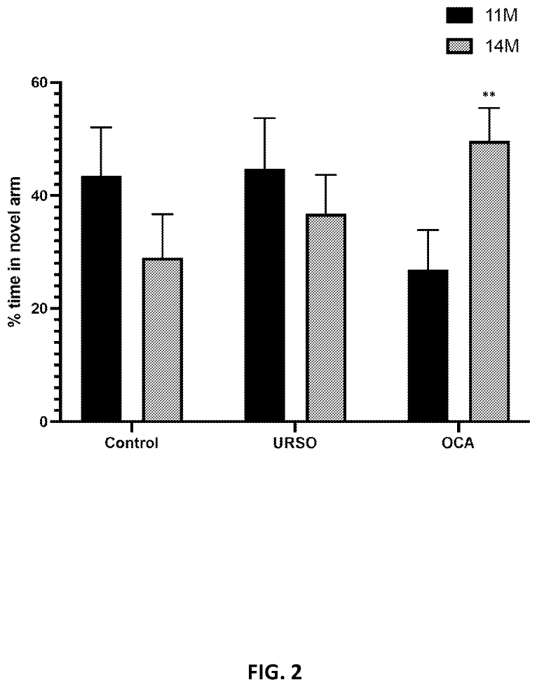

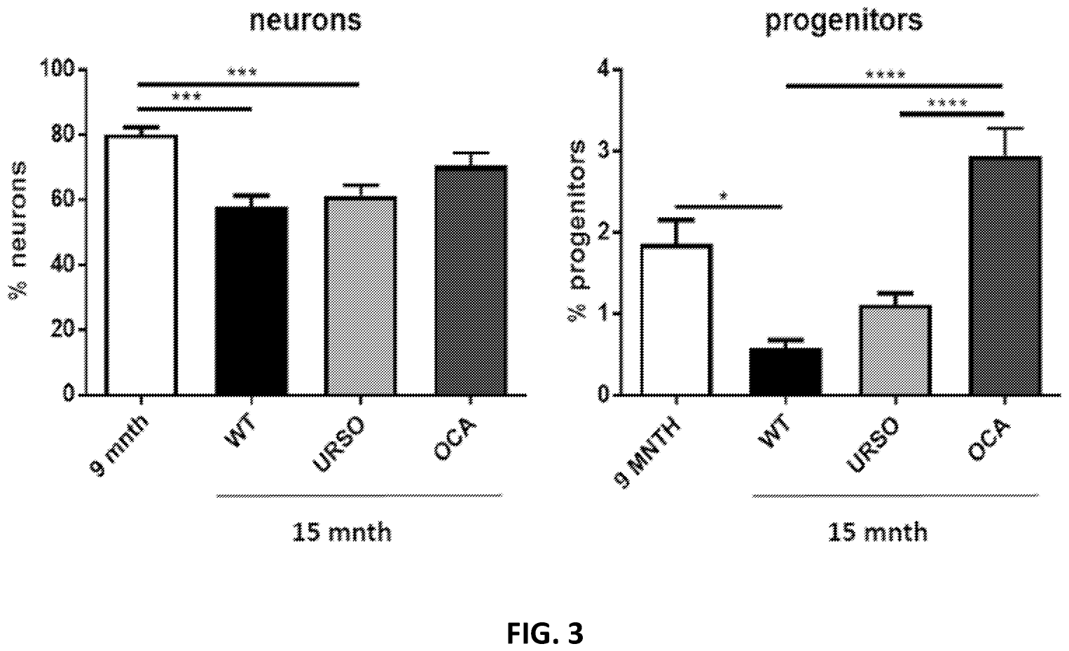

Cognitive impairment is a clinical issue with age-related cognitive decline being a substantial problem in society. The study plan for an aged mice cohort study is shown in FIG. 1. C57 Blk/6 mice were aged to 9 months when they started either 6 months of Ursodeoxycholic acid or Obeticholic acid therapy or were left to age naturally to 15 months. Between 9 and 15 months of age mice develop progressive cognitive decline, by 15 months, with a novel arm time of one third indicating complete loss of short-term spatial memory (FIG. 2). Y-maze testing was repeated on the animals in each group at 11 and 14 months (FIG. 2; left bars and right bars respectively). In both control and Ursodeoxycholic acid groups spatial memory was reduced with age. However, in the obeticholic acid group performance was significantly improved between ages 11 and 14 months. As shown, this effect is completely abrogated by OCA, but not UDCA therapy when given between 9 and 15 months of age in the diet. The effect was not only behavioral. OCA use was again associated with significant increase in neuronal precursors and, in these longer-term experiments, a reversal in overall neuronal cell loss. FIG. 3 shows immunofluorescent analysis of animals, fed ad lib, and given obeticholic acid, ursodeoxycholic acid, or no supplement from aged 9 months to 15 months. Without wishing to be bound by theory these results appear to shown that OCA slows neuronal loss and significantly increases neurogenesis in wild-type (WT) mice. NeuN+ neuron count showed significant loss of neurons in wild type untreated mice and URSO treated mice between 9 and 15 months. There was no significant neuron loss between 9 and 15 months in the OCA supplemented mice, suggesting the protective effect of OCA seen in previous studies also applies to aged but healthy animals. OCA treated animals also had a significant increase on the SOX2+ neural progenitor population compared to both aged matched groups, something which may be contributing to healthy brain aging in these animals and impact neuron retainment and, therefore spatial memory. Non-cholestatic cognitive decline in aged mice used as an experimental model was completely abrogated by OCA, but not UDCA therapy. OCA use was associated with significant increase in neuronal precursors and, in these longer-term experiments, a reversal in overall neuronal cell loss.

### Example 2: In Vitro Blood Brain Barrier (BBB) Modelling in PD

In order to validate the role played by FXR in BBB physiology, we extend an existing three-layer model system with endothelial cells, pericytes, and astrocytes layered over each other in a published transwell model system (see e.g., Hind, W. H. (2014). The Effects of Cannabinoids on Blood-Brain Barrier Permeability in vitro. Ph.D. thesis, University of Nottingham, Nottingham; Wang, Y., et al, (2015). In vitro model of the blood-brain barrier established by co-culture of primary cerebral microvascular endothelial and astrocyte cells. Neural Regen. Res. 10, 2011-2017; He, Yarong, et al. “Cell-culture models of the blood-brain barrier.” Stroke vol. 45, 8 (2014): 2514-26), which has been established within our laboratories. This model allows exposure of endothelial cells to intervention/therapeutic modification and determination of transfer of markers into the astrocyte or pericyte layer and receiver fluid above this. In a PD model, cells derived from iPSC from patients with and without cognitive symptoms, conditioned in culture containing serum from patients with and without cognitive deficits (a four-way experimental design for each disease group). BBB integrity will be assessed by study of transendothelial magnetic resistance EM and direct permeability assay using fluorescent-labelled dextran. The ability of FXR agonists to modify permeability will be directly assessed and the role played by FXR directly will be confirmed using FXR gene knockdown.

### Example 3: Understanding the BBB in Incident PD

Measures of BBB function are performed on the stored cerebrospinal fluid (CSF)/matched plasma of an historic cohort of deeply phenotyped early PD participants, to explore the utility of CSF as a response measure in the clinical evaluation of FXR agonist treatment.

### Example 4: Clinical Study

The clinical study investigates the potential for OCALIVA in patients with PD, using OCALIVA at a dose of 25 mg/day.

Study Population: Diagnosis of PD-MCI according to Movement Disorder Society Level 1 criteria (32); as operationalized by subjective or objective evidence of cognitive decline with no limitation on activities of daily living, plus Montreal Cognitive Assessment (MoCA) score of

Group size: Given the novel dimension to a trial in PD the group numbers are empirical at 25 (i.e. 15 receiving OCALIVA and 10 placebo). There will be an interim safety analysis after the first 10 patients have had a minimum of 4 weeks therapy.

TABLE 1
Trial Summary

| Summary of | Double blind, placebo controlled trial of the FXR agonist Obeticholic Acid | |
|---|---|---|
| Trial Design | (OCA) for the treatment of cognitive dysfunction and related symptoms in | |
| | patients with Parkinson's Disease (PD), at a dose of 25 mg per day according to | |
| | tolerability and identical placebo | |
| Summary of | Patients with Parkinson's Disease | |
| Participant | | |
| Population | | |
| Planned | Total n = 25, (Treatment n = 15; Placebo n = 10) | |
| Sample Size | | |
| Treatment | 26 weeks | |
| Duration | | |
| Follow Up | 4 weeks post treatment | |
| Duration | | |
| | Objectives | Endpoint/Outcome Measures |
| Primary | To assess safety and | Safety and tolerability of obeticholic |
| | tolerability of OCA treatment | acid |
| | in patients with PD. | Number of adverse events |
| | | Number of Serious Adverse |
| | | Events (SAEs) |
| Secondary | To assess the impact of OCA | Cognitive symptom impact |
| | treatment on cognitive | PD-Cognitive Functional Rating |
| | dysfunction in patients with | Scale (PD-CFRS) (change from |
| | PD | baseline to 12 and 26 weeks). |
| | | Symptom domains relevant to |
| | | cognitive symptoms in PD |
| | | Hospital Anxiety and Depression |
| | | Scale (HADS) |
| | | The shortened Neuropsychiatric |
| | | Inventory (NPI-4) |
| | | Epworth Sleepiness Scale (ESS) |
| | | Fatigue (FACIT-F). |
| | | Pittsburgh Sleep Quality Index |
| | | (PSQI) (change from baseline to |
| | | 12 and 26 weeks) |
| | | Global quality of life and health |
| | | utility |
| | | EQ-5D-5L |
| | | PDQ-39. |
| | | Change in other cognitive scores |
| | | MoCA |
| | | Verbal Fluency (change from |
| | | screening to 26 weeks) |
| | To assess the impact of OCA | Change from screening to week 26: |
| | treatment on activities of daily | Movement Disorder Society Unified |
| | living and motor disease | Parkinson's disease rating Scale |
| | | (MDS-UPDRS) parts 2 & 3 |
| | To assess the acceptability of | Completeness of outcomes: |
| | study design | Proportion of participants |
| | | completing study treatment and |
| | | follow up |
| | | Rate of withdrawal and treatment |
| | | discontinuation |
| | | Acceptability of study design to |
| | | participants |
| | | Proportion of invited participants |
| | | who decline trial |
| | | Post-trial questionnaire |
| | To assess health status and | Health status and response to trial |
| | response to medication | medication assessed by the post-trial |
| | | questionnaire completed at 26-weeks |
| | | (End of therapy (EoT) visit) |
| Exploratory | To assess the impact of OCA | Change from baseline to 26 weeks |
| Analyses | treatment on cognitive | using the CANTAB cognitive testing |
| | dysfunction in patients with | listed below: |
| | PD. | Rapid Visual Information |
| | | Processing (RVP)* |
| | | Paired Associates learning |
| | | (PAL)* |
| | | Spatial Working Memory |
| | | (SWM)* |
| | | One Touch Stockings of |
| | | Cambridge (OTS)* |
| | | Reaction Time (RTI) |
| | | Multitasking Test (MTT) |
| | | Emotion Recognition Task |
| | | (ERT) |
| | | *these tasks give a composite |
| | | score |
| | To assess the impact of OCA | Axivity package as digital mobility |
| | treatment on gait and physical | outcomes measures: |
| | activity in patients with PD. | Macrostructural (macro) gait |
| | | characteristics (volume, |
| | | variability, pattern of walking |
| | | bouts and steps) |
| | | Microstructural (micro) gait |
| | | characteristics contained |
| | | within each walking bout |
| | | (discrete gait features related |
| | | to phases of the gait cycle and |
| | | other signal characteristics) |
| | | (change from screening to 26 weeks) |
| | To assess the impact of OCA | Clinical Global Impression of Change |
| | treatment on improvement in | Clinician Global Rating Scale |
| | cognitive function in patients | Clinical Rating of Change - |
| | with PD. | Clinician and Carer Observer |
| | | version |
| | | (change from baseline to 26 weeks) |
| Experimental | To assess the impact of OCA | MRI Imaging parameters |
| Imaging | treatment on mechanistic brain | Diffusion Tensor Imaging (DTI) |
| Endpoints | changes in patients with PD. | T1 mapping |
| | | (change from baseline to 26 weeks) |
| | To determine whether OCA | Structural (3D T1w) imaging |
| | has any impact on brain | Change from baseline to 26 |
| | morphology over the duration | weeks in regional grey matter |
| | of the treatment period. | volume. |
| | To determine the role of white | T2w FLAIR imaging |
| | matter lesion volume on | Total volume of white matter |
| | cognitive function. | hyperintensities on T2w |
| | | FLAIR imaging |
| | To determine whether OCA | Resting state functional MRI scan |
| | treatment leads to changes in | Change from baseline to 26 |
| | patterns of brain network | weeks in default mode resting |
| | activity. | state network activity. |
| | To determine whether OCA | Changes from baseline to 26 weeks in |
| | treatment leads to changes | NAA concentration (Proton MRS |
| | neuro-axonal integrity/ | measurement of NAA) |
| | mitochondrial function | |

### Eligibility Criteria

Eligibility must be assessed by a medically qualified doctor and this assessment documented in the participant's medical notes. Only personnel formally delegated by the Principal Investigator to assess eligibility may perform this task.

### Inclusion Criteria

Diagnosis of idiopathic Parkinson's disease made by a movement disorder specialist; Hoehn and Yahr stage I to III
Diagnosis of PD-MCI according to Movement Disorder Society Level 1 criteria (Litvan I et al., Diagnostic criteria for mild cognitive impairment in Parkinson's disease: Movement Disorder Society Task Force guidelines. Mov Disord. 2012 March; 27(3):349-56); as operationalised by subjective or objective evidence of cognitive decline with no limitation on activities of daily living, plus Montreal Cognitive Assessment (MoCA) score of <26/30. The MoCA has been recommended as a scale for use in all types of PD studies, including treatment trials (Matej Skorvanek et al., Reply: MoCA for cognitive screening in Parkinson's disease: Beware of floor effect. Mov Disord. 2018 March; 33(3):499-500)
High risk of unfavourable prognosis (>50% risk of progression to postural instability, dementia or death at 5 years using model developed by Velseboer 2016)
Ability to take study drug
Good command of the English language (to ensure that participants are able to comply with cognitive testing)
Ability to consent, able to comply with study protocol and attend clinic visits
For women of childbearing potential: Willing to use highly effective contraception or to practice sexual abstinence to avoid pregnancy for entire duration of treatment period and for 3-days after.
Age >18 and <80
   The inclusion criteria have been formulated to generate a study population with evidence of mild cognitive impairment but no dementia.
   Abstinence is acceptable only as true abstinence: when this is in line with the preferred and usual lifestyle of the patient. [Periodic abstinence (e.g., calendar, ovulation, symptothermal, post-ovulation methods) and withdrawal are not acceptable methods of contraception].

### Exclusion Criteria

Known or suspected cirrhosis of the liver indicated by a liver biopsy confirming cirrhosis, a history of oesophageal varices, hepatic encephalopathy or ascites, an ultrasound scan suggesting portal hypertension, fibroscan with a reading of >17 KPa or a platelet count <150,000 without an alternative explanation.
Current use of cholestyramine or any other bile-acid binding resin
Elevation of ALT or AP over two times upper limit of normal (ULN)
Bilirubin >1.5×ULN
Inter-current disease characterised by cognitive dysfunction (dementia, delirium or neurodegenerative diseases including multiple sclerosis, motor neurone disease, atypical parkinsonian disorders (multiple system atrophy, progressive supranuclear palsy, corticobasal degeneration).)
Inter-current medication which may worsen cognitive dysfunction (opiates other than codeine phosphate, sleeping pills (with the exception of melatonin and clonazepam for management of REM sleep behaviour disorder), regular (daily) anti-histamine use (in the last four weeks), anti-psychotic agents or recreational drug use)
Use of cholinesterase inhibitors, memantine, antipsychotics, amantadine, or modafanil within the last 3 months.
Anticipated cognitive medication change within the duration of the study (addition of cholinesterase inhibitor or memantine) or taken within the last 3 months.
Contraindications to MRI assessment (active medical implants, e.g., Cardiac pacemaker, metal implants)
Previous exposure to OCA (either in clinical trials or in clinical practice) or Fibrate therapy for ≥3 months and within the last 3 months
History of active malignant disease in the last 5 years
Atypical parkinsonian syndromes (including multiple system atrophy, progressive supranuclear palsy, corticobasal degeneration); drug-induced parkinsonism; vascular parkinsonism; essential tremor
Previous deep brain stimulation surgery for PD; previous gene therapies or cell therapies
Regular (more than one week per month) alcohol consumption in excess of recommended safe limits (14 units per week). Excess alcohol consumption within the last 5 years
Pregnancy or planning to get pregnant during duration of treatment period
Any medical or psychiatric condition that may impair ability to participate
Patients with a dementia diagnosis outlined by DSM V criteria; or fulfilled Parkinson's disease dementia (PDD) criteria set by the Movement Disorder Society (Emre et al Clinical diagnostic criteria for dementia associated with Parkinson's disease. Mov Disord. 2007 Sep. 15; 22(12):1689-707)
Active participation in another interventional trial or exposure to another experimental drug within 5 half-lives.
Patients who do not have the capacity to consent to be involved in the study (as assessed by criteria laid out in the MHA code of practice, section 4-3).

Randomization: Patients who provide informed consent to participate and fulfill the eligibility criteria will be randomly allocated to receive either obeticholic acid or matched placebo by designated and trained members of the research team using central, secure, web-based randomization with concealed allocation. Patients will be randomized in a ratio of 1:1.5 (placebo: intervention). The allocation sequence will be computer-generated, using a random permuted block design, with blocks of varying sizes. Block sizes will not be disclosed, to ensure concealment.

### Trial Assessments:

Medical History: A background medical history (including history of falls in the previous year, history of REM sleep behaviour disorder and any visual hallucinations) plus medication history will be obtained.

Clinical Examination: A full physical examination will be conducted. It will include examination of hair and skin, lymph nodes; eyes; ears; nose; throat; respiratory; cardiovascular; abdomen; musculoskeletal; neurological assessment; mental status assessment. Bodyweight will also be measured.

Vital signs: Pulse, blood pressure and temperature will be recorded.

ECG: A standard 12 lead ECG will be taken and assessed at screening (visit 1), Interim Visit 6 (12 weeks) and at the End of Therapy visit 9 (26 weeks).

Blood Sampling: A blood sample will be taken (maximum 25 ml) at times specified in the schedule of events (Table 3) for the following assessments:
Kidney Function test: urea, creatinine and serum electrolytes
Liver Function test: ALT, AST, AP, total bilirubin, direct bilirubin, GGT, LDH
Cholesterol (including LDL, HDL)
Full blood count (hemoglobin, platelets & white cells—neutrophils & lymphocytes)

Pregnancy testing: For sexually active female participants of childbearing potential a urine sample based pregnancy test will be carried out during the trial screening visit.

### Questionnaire Based Symptom Assessment Tool Package:

EQ-5D: EQ-5D is a 5 item, validated general quality of life measure form which health utility can be calculated. This is a score within the range 0-100 for quality of life, where 0 is equivalent to death and 100 represents perfect health. EQ-5D is optimized for patient self-completion and will be undertaken on paper in this study. Participants are asked to describe their health as of today.

PDQ-39: The Parkinson's Disease Quality of Life Questionnaire (PDQ-39) (Peto V, Jenkinson C, Fitzpatrick R, Greenhall R. The development and validation of a short measure of functioning and well being for individuals with Parkinson's disease. Qual Life Res. 1995; 4(3):241-8) is used as a measure of global quality of life (QoL) has excellent internal consistency and test-retest validity, and, furthermore, it is sensitive to change over time, making it suitable for use in longitudinal studies. It has been validated for use in PD and has been widely adopted. Comprised of 39 items, it assesses health related quality of life (HRQoL) across eight domains encompassing mobility (10 items), ADLs (six items), emotional well-being (six items), stigma (four items), social support (three items), cognition (four items), communication (three items), and bodily discomfort (three items). Responses are recorded on a scale ranging from 0 (never) to 4 (always). The score from each subscale is transformed into a 0 to 100 score by summing the raw score from each item, dividing by the maximum possible raw score, and multiplying by 100. A composite summary index score (PDQ-SI) of the eight domains provides an overall measure of the impact of illness on functioning and well-being that reduces the role of chance, which may occur when exploring individual PDQ-39 domains. This is calculated using the mean of each domain score: Higher scores indicate poorer perceived health. Participants are asked to rate their activities during the last month.

FACIT-F: The Functional Assessment of Chronic Illness Therapy-Fatigue (FACIT-F) scale was developed from interviews with oncology patients and clinical experts to assess anemia-associated fatigue in the past 7 days. It is a patient-reported rating scale consisting of 13 items (statements) with five ordered response categories (“not at all”-“a little bit”-“somewhat”-“quite a bit”-“very much”) regarding the respondents' situation during the past week. It yields a summed total score ranging between 0 and 52 (52=less fatigue). Item contents cover the experience (e.g., feelings of tiredness, listlessness, energy) as well as the impact (e.g., trouble doing things, need to sleep, social limitations) of fatigue but does not provide a definition of fatigue. It has been used and validated in a range of patient groups, such as RA, PD, various forms of cancer, and in the general population.

PITTSBURGH Sleep Scale: The Pittsburgh Sleep Scale/Pittsburgh Sleep Quality Index is a self-reported questionnaire to evaluate the quality of sleep over one month period. It includes 19 items which are used to create seven components (subjective sleep quality, sleep latency, sleep duration, habitual sleep efficiency, sleep disturbances, use of sleeping medication and Daytime dysfunction). Each component has score 0-3 with possible score ranging from 0-21 with lower scores denoting a healthier sleep quality. Pittsburgh Sleep Quality Index will be undertaken on paper in this study.

ESS: The Epworth Sleep Scale (ESS) is a short questionnaire to measure daytime sleepiness in recent times. It includes 8 questions with each answer rated from 0 to 3. Possible results range from 0 to 24. Scores below 9 are considered to be in normal range while scores equal to and more than 9 may need specialist assessment for further investigation and treatment. ESS is optimized for patient self-completion and will be undertaken on paper in this study.

HADS: The Hospital Anxiety and Depression Scale (HADS) is a 14 items measurement tool to assess anxiety and depression. Each item scores from 0 to 3. Seven of the items relate to anxiety domain and the other seven items are for depression domain. Scores of eleven or greater than eleven on each symptom domain facilitate the identification of the symptom. HADS is optimised for patient self-completion and will be undertaken on paper in this study.

A total score greater than 10 on the ‘A’ (odd numbered) questions (indicating abnormal or uncontrolled levels of anxiety) or on the ‘D’ (even-numbered) questions (indicating abnormal or uncontrolled levels of depression) will trigger the offer of a clinical review by a study doctor and feedback to GP

NPI-4: The Neuropsychiatric Inventory Brief Measure (NPI-4) assesses presence of psychopathology in patients with brain disorders over the past month. Ten behavioural (delusions, hallucinations, agitation/aggression, depression, anxiety, elation/euphoria, apathy/indifference, disinhibition, irritability, aberrant motor behaviour) and two neurovegetative areas (sleep and night time behaviour disorders, appetite and eating disorders) are included in the NPI. The NPI is based on responses from an informed caregiver, preferably one living with the patient. If an informed observer is not available, the instrument cannot be used or must be modified. In a previous analysis of NPI baseline scores in a sample of dementia with Lewy body patients, a four item subscore calculated as the sum of the scores of delusions, hallucinations, apathy, and depression was identified as the main Lewy-body symptoms cluster. This subscore (NPI-4) has subsequently been used as primary outcome criterion in interventional studies. In the NPI-4, informants are asked about the presence or absence of behavioural changes for each subscore. If present, the frequency and severity are determined, and a composite score obtained for each item (maximum 12). The NPI-4 total is then summed from each of the composite scores (maximum 48). Higher scores indicate a greater disturbance of behaviour.

PD-CFRS: The Parkinson's Disease Cognitive Functional Rating Scale (PD-CFRS) is a five minute PD-specific questionnaire which explores a wide range of functional aspects suspected to be sensitive to cognitive impairment in PD, minimising the motor impact of the disease. It measures the functional impact of cognitive impairment in PD and change over time. It can be repeated after six months, at which time an increase of two points indicates a clinically significant worsening of the cognitive functional status. It is not subject to confounding by motor disability, age, education, anxiety, or depression. It is valid, reliable, and offers a reasonable sensitivity of 84% and specificity of 69% at a cut-off of greater than two to detect PD with mild cognitive impairment (PD-MCI) with associated functional impairment. It can also diagnose PD dementia (PDD) with a sensitivity and specificity of 83% using a higher cut-off value of greater than six.

Participants are asked 12 questions on how cognitive difficulties have hindered their daily life of the past two weeks (score 0-24).

This questionnaire may also be completed by a knowledgeable informant, but for consistency should be the same person for the participant within the study, for example if it was the carer at baseline, it should be the carer at subsequent visits. There is a carer information sheet and consent form to be completed so that they can answer this questionnaire.

CANTAB: Measure scores for the Cambridge Neuropsychological Test Automated Battery (CANTAB) cognitive testing platform will be collected at baseline and at week 26.

The following domains listed in the table below (Table 2 CANTAB tasks) will form the exploratory outcome.

TABLE 2
CANTAB tasks

| Area | CANTAB Task | Acronym |
|---|---|---|
| Attention and Psychomotor Speed | Rapid Visual Information Processing | RVP* |
| Memory | Paired Associates learning | PAL* |
| Memory | Spatial Working Memory | SWM* |
| Executive Function | One Touch Stockings of Cambridge | OTS* |
| Attention and Psychomotor Speed | Reaction Time | RTI |
| Executive Function | Multitasking Test | MTT |
| Social and Emotion Cognition | Emotion Recognition Task | ERT |

*These tasks will also give a composite score value at trial end.

MDS-UPDRS Part II ((including Hoehn and Yahr Stage): The Movement Disorder Society revised Unified Parkinson's Disease Rating Scale (MDS-UPDRS) Part III is based on examination of the participant and is a measure of motor severity (range 0-132). Each parkinsonian sign is rated on a 5-point Likert scale (ranging from 0 to 4), with higher scores indicating a more severe impairment. This should be performed whilst the participant is “on”—the typical functioning state when patients are receiving medication and have a good response.

Hoehn and Yahr Stage is a long standing internationally established scale that mirrors disability of patients and is as below:
Stage 0 No signs of disease
Stage 1 Unilateral disease
Stage 2 Bilateral disease, without impairment of balance
Stage 3 Mild to moderate bilateral disease; some postural instability and needs assistance to recover from the pull test; physically independent
Stage 4 Severe disability; still able to walk or stand unassisted
Stage 5 Wheelchair bound or bedridden unless aided.

PD-CFRS: The Parkinson's Disease Cognitive Functional Rating Scale (PD-CFRS) is a five minute PD-specific questionnaire which explores a wide range of functional aspects suspected to be sensitive to cognitive impairment in PD, minimising the motor impact of the disease. It measures the functional impact of cognitive impairment in PD and change over time. It can be repeated after six months, at which time an increase of two points indicates a clinically significant worsening of the cognitive functional status. It is not subject to confounding by motor disability, age, education, anxiety, or depression. It is valid, reliable, and offers a reasonable sensitivity of 84% and specificity of 69% at a cut-off of greater than two to detect PD with mild cognitive impairment (PD-MCI) with associated functional impairment. It can also diagnose PD dementia (PDD) with a sensitivity and specificity of 83% using a higher cut-off value of greater than six.

The scale is administered to a knowledgeable informant in interview form by 12 items selected to cover the spectrum of instrumental cognitive changes seen in PD over the last two weeks before the evaluation. All 12 questions explore with some examples, whether or not the patient has had trouble in performing an activity (0=none; 1=some of the time; 2=most of the time; 8=the subject has never done the activity in the past) such as handling money, domestic economy, arranging holidays or meetings, handling personal mail, controlling drug treatment schedule, organizing daily activities, handling home electrical appliances, understanding how to use public transport, solving unforeseen events, explaining things he/she want to say, understanding the things he/she read, and handling the cell phone. The maximum score, obtained by the sum of the ratings, is 24. Whenever there are “eights” in the record sheet indicating an activity never done in the past, the mean of all the items answered 0-2 is calculated, and this mean value (either 0, 1 or 2) is scored in those items answered with an eight. (J. Kulisevsky et al. Parkinsonism and Related Disorders (2013) 19: 812-817).

Verbal Fluency: Participants are asked to name as many words as they can beginning with the letter ‘F’ in one minute (item taken from MoCA). Semantic fluency will be assessed with the animal fluency test, in which patients are asked to name as many animals as possible in a 90 second time frame.

MOCA: The Montreal Cognitive Assessment is a measure of global cognitive function (maximum 30). Scores <26 may indicate a degree of impaired cognition.

Clinical Global Impression of Change: The modified clinical global impression of change (CGIC) will be scored following baseline interview/review of assessments and review at 26 weeks. At baseline, notes will be made with respect to history, observation, attention, orientation, memory, language praxis, judging, behaviour and function. This will be repeated at 26 weeks and scored as 1 (very much better), 2 (much better), 3 (slightly better), 4 (no change), 5 (slightly worse), 6 (much worse), 7 (very much worse).

This questionnaire will be completed by a knowledgeable informant/carer as well as by a clinician. For consistency the informant/carer and clinician should be the same person for the participant within the study, for example if it was the carer at baseline, it should be the carer at 26 weeks visits. There is a carer information sheet and consent form to be completed so that they can answer this questionnaire.

Axivity Package: Ambulatory activity and gait will be measured in the home using a small, lightweight and waterproof monitor (Axivity AX6; Axivity Ltd, UK). The wearable sensor worn on the lower back (fifth lumbar vertebrae), with a sampling frequency of 100 Hz. Outcomes measured will be in keeping with published work and will include a brief postural control and gait assessment. This will be worn for seven days and will provide free-living data in terms of activity, gait and functional performance.

Macrostructural (behavioral) characteristics that will be calculated from the activity package will include the volume, pattern and variability (S₂) of walking. Volumetric outcomes will include total walking time per day, percentage (%) of walking time per day, number of bouts and steps per day. Pattern will include mean bout length, generated based on the ambulatory bout (AB) detected over the 7 days, and a non-linear descriptor (alpha (a)). Alpha describes ABs distribution, evaluating the ratio of short to long ABs (i.e. a high alpha means that the total walking time is made up of proportionally short ABs compared to long ABs). Macrostructural gait variability (S₂) will be derived evaluating the ‘within subject’ variability of AB length, with a higher Macro gait variability (S₂) indicating a more varied walking activity pattern, while a lower Macro gait variability (S₂) would mean a less varied walking activity, so a reduced engagement in different activity and a tendency to repeat the same pattern of activity.

Microstructural gait characteristics (n=14) will be determined for each AB. Micro characteristics were selected based upon a model of gait comprising five domains (pace, variability, rhythm, asymmetry and postural control) validated both in older adults and in PD. Briefly, the initial contact and final contact events within the gait cycle will be identified and allow the estimation of step, stance and swing time. Initial contact events will be also used to estimate step length using the inverted pendulum model. Step velocity will be calculated as the ratio between step length and time. To evaluate micro gait variability (e.g., step time variability), the standard deviation (SD) from all steps (left and right combined) will be calculated. Asymmetry will be determined as the absolute difference between left and right steps for each AB, averaged across all ABs.

Magnetic resonance imaging (MRI): The following MRI protocol will be used for all relevant trial visits as described in the schedule of events:
Structural (3D T1w) imaging—to evaluate disease-related differences in brain macrostructure, particularly in grey matter.
T2w FLAIR imaging—to identify any areas of gross white matter disease via quantification of white matter hyperintensities (WMH).
Diffusion Tensor Imaging (DTI)—for tract based spatial statistical (TBSS) analysis of white matter tract changes, building on the highly significant correlation between white matter fractional anisotropy and functional measures in pilot PBC studies.
T1 mapping—to provide quantitative values on tissue properties, included as a metric to seek subtle reversible changes in the grey matter.
Resting state functional MRI scan—to measure interconnection of cortical brain areas which form networks based on correlated slowly-varying brain activity
Proton MRS Measurement of NAA Concentration—assessed in a single 8 cm³ volume placed in white matter to measure neuro-axonal integrity, which can be altered due to abnormal mitochondrial function and/or to white matter tract changes (see also DTI).

All scans will be conducted on a single MRI instrument at Newcastle University MR Centre using a 3T Philips Achieva scanner with 8 channel head coil and can be collected within the duration of a standard neuro MRI investigation (less than 1 hour). All scans will be collected using advanced versions of sequences which are available on all modern MR scanners.

TABLE 3
Schedule of Events:

| | Assessment | | | | | | | | | | |
|---|---|---|---|---|---|---|---|---|---|---|---|
| | | | Interim | Interim | Interim | | Interim | Interim | End of | Resolu- | |
| | | Baseline | Visit | Visit | telephone | Interim | telephone | telephone | therapy | tion | |
| | Screening | (Visit 2) | (Visit 3) | (Visit 4) | Visit | Visit | Visit | Visit | Visit | Visit | ***Reso- |
| | (Visit 1) | Day 0 | 2 weeks | 4 weeks | (Visit 5) | (Visit 6) | (Visit 7) | (Visit 8) | (Visit 9) | (Visit 10) | lution |
| | >Day −7 | (+/−1 | (+/−1 | (+/−1 | 8 weeks | 12 weeks | 16 weeks | 20 weeks | 26 weeks | 30 weeks | phone |
| | to Day −1 | wk) | wk) | wk) | (+/−1 wk) | (+/−1 wk) | (+/−1 wk) | (+/−1 wk) | (+/−1 wk) | (+/−1 wk) | call |
| Informed Consent | X | | | | | | | | | | |
| Demographics & | X | | | | | | | | | | |
| medical history | | | | | | | | | | | |
| Clinical examination | X | | | | | | | | X | | |
| ECG | X | | | | | X | | | X | | |
| Vital signs | X | | X | X | | X | | | X | X | |
| Blood sampling | X | | X | X | | X | | | X | X | |
| Kidney function | | | | | | | | | | | |
| (Urea + Electrolytes) | | | | | | | | | | | |
| Liver function test | | | | | | | | | | | |
| (LFT) | | | | | | | | | | | |
| Cholesterol | | | | | | | | | | | |
| Full Blood Count | | | | | | | | | | | |
| Biobank blood | | X | | | | X | | | X | | |
| sample (optional) | | | | | | | | | | | |
| Biobank blood | | X | | | | | | | X | | |
| sample (optional) | | | | | | | | | | | |
| Inflammatory markers | | | | | | | | | | | |
| Pregnancy testing | X | | | | | | | | | | |
| (females of child- | | | | | | | | | | | |
| bearing age only) | | | | | | | | | | | |
| Eligibility assessment | X | | | | | | | | | | |
| Randomization* | X | | | | | | | | | | |
| 7 day activity | X | | | | | | | | X | | |
| monitoring (Axivity) | | | | | | | | | | | |
| MDS-UPDRS | X | | | | | | | | X | | |
| (part 3) | | | | | | | | | | | |
| Verbal Fluency**** | X | | | | | | | | X | | |
| MOCA | X | | | | | | | | X | | |
| Clinical Global | | X | | | | | | | X | | |
| Impression of Change | | | | | | | | | | | |
| Questionnaire based | | X | | | | X | | | X | X | |
| symptom assessment | | | | | | | | | | | |
| tool package: | | | | | | | | | | | |
| EQ-5D-5L, PDQ 39, | | | | | | | | | | | |
| FACIT-F, PSQI, | | | | | | | | | | | |
| ESS, HADS, NPI-4, | | | | | | | | | | | |
| PD CFRS | | | | | | | | | | | |
| Post-trial | | | | | | | | | X | | |
| questionnaire | | | | | | | | | | | |
| CANTAB - | X | | | | | | | | | | |
| Familiarization test | | | | | | | | | | | |
| CANTAB | | X | | | | | | | X | | |
| MRI | | X | | | | | | | X | | |
| Treatment | | X | X | X | | X | | | | | |
| prescription** | | | | | | | | | | | |
| Compliance***** | | | X | X | | X | X | X | X | | |
| Concomitant | X | X | X | X | | X | X | X | X | | |
| medication | | | | | | | | | | | |
| Adverse event | X | X | X | X | X | X | X | X | X | X | X |
| reporting | | | | | | | | | | | |

*Participant can be randomized prior to baseline visit, once all screening activity has been completed and participant has been deemed eligible by the PI or other medically qualified delegated person at site.
**Where possible and where aligned with local process prescriptions may be generated and sent to pharmacy one working day ahead of scheduled participant visit.
***Only for participants who have stopped taking their trial medication and have either withdrawn completely or over 4 weeks from their next trial visit to check on any safety issues/adverse events they may experience in the wash out period. Participants will be asked if they consent to this as part of the Withdrawal/discontinuation of treatment process.
****Verbal Fluency and MOCA assessments performed at screening visit and therefore do not need to be repeated at baseline visit.
*****Site staff to check drug diaries for patient compliance with treatment. Tablet count to be performed by site staff at weeks 12 and 26.

Dosage Schedule & Modifications: For all participants initiation of trial treatment will commence once screening tests are complete and participant attends baseline visit. From baseline, participants will receive 25 mg once a day of obeticholic acid or Placebo to be taken orally.

If issues with tolerability are encountered participants will be offered dose reduction using a predefined schedule:

25 mg daily: Where patients are taking 25 mg obeticholic acid/placebo daily (one tablet of the trial medication) dose reduction should be to 25 mg/placebo (i.e. one tablet) on alternate days.

If tolerability is still an issue patients should reduce to 25 mg/placebo (i.e. one tablet) once per week.

At least a week should be allowed to elapse between dose reductions.

Participants may experience side effects that mean that they wish to stop taking the trial medication. Participants can have up to a one week break from the medication (7 days) once while taking the trial medication.

1. A method of ameliorating, mitigating, treating, or preventing cognitive impairment in a subject, comprising administering to the subject at least one therapeutically effective amount of a compound of formula (I): 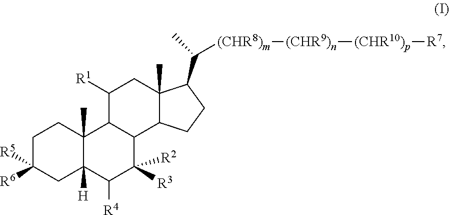
or a pharmaceutically acceptable salt, ester, solvate, or amino acid conjugate thereof, wherein:
R¹ is H, OH, alkoxy, or oxo;
R² is H, OH, halogen, or alkyl optionally substituted with one or more halogen or OH, or R² and R³ taken together with the carbon atom to which they are attached form a carbonyl;
R³ is H, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, or halogen, or R⁵ and R⁶ taken together with the carbon atom to which they are attached form a carbonyl;
R⁷ is OH, OSO₃H, SO₃H, OSO₂NH₂, SO₂NH₂, OPO₃H₂, PO₃H₂, CO₂H, C(O)NHOH, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, or 2,4-difluoro-3-hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
m is 0, 1, or 2;
n is 0 or 1; and
p is 0 or 1.

2. The method of claim 1, wherein the compound of formula (I) is a compound of formula (A): 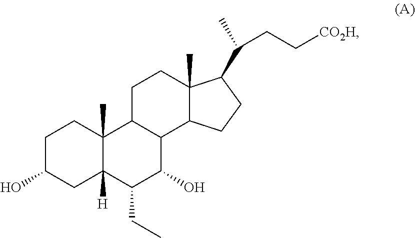 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]
or pharmaceutically acceptable salt, ester, solvate, or amino acid conjugate thereof.

3. The method of claim 2, wherein the compound of formula (I) is a compound of formula (A).

4. The method of claim 1, wherein the compound of formula (I) is a compound of formula (B): 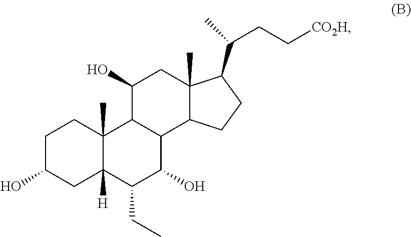 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]
or pharmaceutically acceptable salt, ester, solvate, or amino acid conjugate thereof.

5. The method of claim 4, wherein the compound of formula (I) is a compound of formula (B).

6. The method of claim 1, wherein the compound of formula (I) is a compound of formula (C): 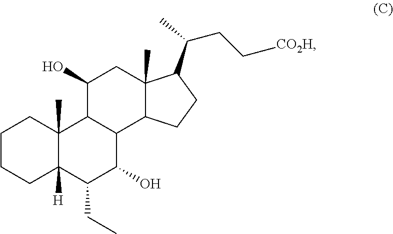
or pharmaceutically acceptable salt, ester, solvate, or amino acid conjugate thereof.

7. The method of claim 6, wherein the compound of formula (I) is a compound of formula (C).

8. The method of claim 1, wherein the compound of formula (I) is a compound of formula (D): 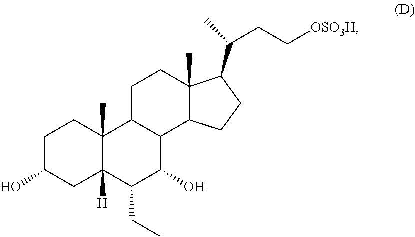
or pharmaceutically acceptable salt, ester, solvate, or amino acid conjugate thereof.

9. The method of claim 8, wherein the compound of formula (I) is a compound of formula (D).

10. The method of claim 1, wherein the cognitive impairment is associated with a neurological disease.

11. The method of claim 10, wherein the neurological disease is Parkinson's disease.

12. The method of claim 10, wherein the neurological disease is Parkinson's disease dementia.

13. The method of claim 10, wherein the neurological disease is mild cognitive impairment in Parkinson's disease dementia (PD-MCI).

14. The method of claim 1, wherein the effective amount of the compound of formula (I) is between about 1 mg and about 50 mg.

15. The method of claim 14, wherein the effective amount of the compound of formula (I) is between about 1 mg and about 25 mg.

16. The method of claim 14, wherein the effective amount of the compound of formula (I) is about 25 mg.

17. The method of claim 1, wherein the effective amount of the compound of formula (I) is administered about every day, about every two days, about every three days, or about every week.

18. The method of claim 17, wherein the effective amount of the compound of formula (I) is administered daily.

19. A method of reversing cellular senescence associated with Parkinson's Disease in the neurons of a subject, comprising administering to the subject at least one therapeutically effective amount of a compound of formula (I) 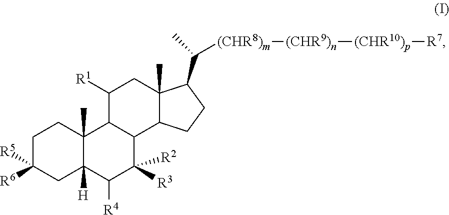
or a pharmaceutically acceptable salt, ester, solvate, or amino acid conjugate thereof, wherein:
R¹ is H, OH, alkoxy, or oxo;
R² is H, OH, halogen, or alkyl optionally substituted with one or more halogen or OH, or R² and R³ taken together with the carbon atom to which they are attached form a carbonyl;
R³ is H, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, or halogen, or R⁵ and R⁶ taken together with the carbon atom to which they are attached form a carbonyl;
R⁷ is OH, OSO₃H, SO₃H, OSO₂NH₂, SO₂NH₂, OPO₃H₂, PO₃H₂, CO₂H, C(O)NHOH, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, or 2,4-difluoro-3-hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
m is 0, 1, or 2;
n is 0 or 1; and
p is 0 or 1.

20. A method of ameliorating, mitigating, treating, or preventing telomere dysfunction associated with Parkinson's Disease in the neurons of a subject, comprising administering to the subject at least one therapeutically effective amount of a compound of formula (I) 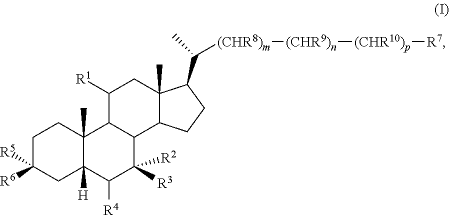
or a pharmaceutically acceptable salt, ester, solvate, or amino acid conjugate thereof, wherein:
R¹ is H, OH, alkoxy, or oxo;
R² is H, OH, halogen, or alkyl optionally substituted with one or more halogen or OH, or R² and R³ taken together with the carbon atom to which they are attached form a carbonyl;
R³ is H, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, or halogen, or R⁵ and R⁶ taken together with the carbon atom to which they are attached form a carbonyl;
R⁷ is OH, OSO₃H, SO₃H, OSO₂NH₂, SO₂NH₂, OPO₃H₂, PO₃H₂, CO₂H, C(O)NHOH, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, or 2,4-difluoro-3-hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
m is 0, 1, or 2;
n is 0 or 1; and
p is 0 or 1.

21-23. (canceled)

24. A kit for treating, ameliorating, or mitigating cognitive impairment in a subject, comprising at least one therapeutically effective amount of a compound of formula (I): 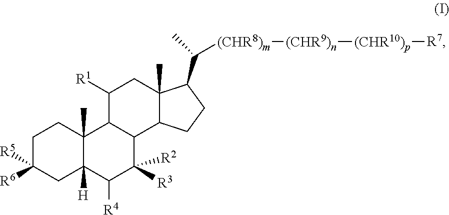
or a pharmaceutically acceptable salt, ester, solvate, or amino acid conjugate thereof, and instructions for use; wherein:
R¹ is H, OH, alkoxy, or oxo;
R² is H, OH, halogen, or alkyl optionally substituted with one or more halogen or OH, or R² and R³ taken together with the carbon atom to which they are attached form a carbonyl;
R³ is H, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, or halogen, or R⁵ and R⁶ taken together with the carbon atom to which they are attached form a carbonyl;
R⁷ is OH, OSO₃H, SO₃H, OSO₂NH₂, SO₂NH₂, OPO₃H₂, PO₃H₂, CO₂H, C(O)NHOH, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, or 2,4-difluoro-3-hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
m is 0, 1, or 2;
n is 0 or 1; and
p is 0 or 1.

25. A method of treating, ameliorating or mitigating cognitive impairment in a subject comprising administering to the subject a pharmaceutical composition comprising at least one therapeutically effective amount of a compound of formula (I): 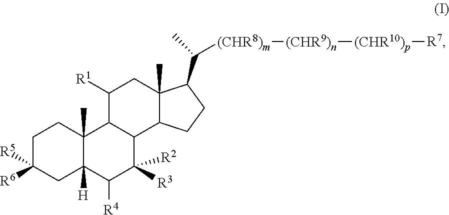
or a pharmaceutically acceptable salt, ester, solvate, or amino acid conjugate thereof, and at least one pharmaceutically acceptable excipient; wherein:
R¹ is H, OH, alkoxy, or oxo;
R² is H, OH, halogen, or alkyl optionally substituted with one or more halogen or OH, or R² and R³ taken together with the carbon atom to which they are attached form a carbonyl;
R³ is H, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, or halogen, or R⁵ and R⁶ taken together with the carbon atom to which they are attached form a carbonyl;
R⁷ is OH, OSO₃H, SO₃H, OSO₂NH₂, SO₂NH₂, OPO₃H₂, PO₃H₂, CO₂H, C(O)NHOH, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, or 2,4-difluoro-3-hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
m is 0, 1, or 2;
n is 0 or 1; and
p is 0 or 1.
